(19) United States
(12) Patent Application Publication
(10) Pub. No.: US 2011/0081416 A1
(43) Pub. Date: Apr. 7, 2011

(54) ORDERED MESOPOROUS SILICA MATERIAL

(75) Inventors: Jasper JAMMAER, Brussels (BE); Alexander Aerts, Borgerhout (BE); Guy Van Den Mooter, Pellenberg (BE); Johan Martens, Huldenberg (BE)

(73) Assignee: FORMAC Pharmaceuticals N.V., Leuven (BE)

(21) Appl. No.: 12/905,759

(22) Filed: Oct. 15, 2010

Related U.S. Application Data
(63) Continuation-in-part of application No. PCT/EP2009/055122, filed on Apr. 28, 2009.
(60) Provisional application No. 61/201,532, filed on Dec. 11, 2008.
(60) Provisional application No. 61/137,673, filed on Aug. 1, 2008.
(60) Provisional application No. 61/125,795, filed on Apr. 28, 2008.
(60) Provisional application No. 61/324,070, filed on Apr. 14, 2010.

(30) Foreign Application Priority Data
Apr. 28, 2008 (GB) 08 076 96.0
Feb. 27, 2009 (GB) 09 033 95.2

(51) Int. Cl. A61K 9/00; C01B 33/12; B32B 3/26

(52) U.S. Cl. 424/484; 423/335; 428/315.7

(57) ABSTRACT

A process for preparing a 2D-hexagonal ordered mesoporous silica material with a substantially uniform pore size in the range of 4 to 30 nm comprising the steps of: preparing an aqueous solution comprising an alkali silicate solution; preparing an aqueous solution comprising a poly(alkylene oxide) triblock copolymer and a buffer with a pH greater than 2 and less than 8, adding said aqueous alkali silicate solution to said aqueous solution giving a pH greater than 2 and less than 8 and allowing a reaction between the components to take place at a temperature in the range of 10 to 100° C., and filtering off, drying and calcinating the reaction product to produce said 2D-hexagonal ordered mesoporous silica material with a substantially uniform pore size. In an alternative procedure both aqueous solutions are fed as liquid streams into an elongated mixing receptacle having a first and a second opening such that the liquid streams of the solutions are each discharged independently into said first opening of said elongated mixing receptacle such that they directly impinge thereby giving a pH in the resulting mixture of greater than 2 and less than 8 and producing a reaction product at a temperature in the range of 10 to 100° C. in said elongated mixing receptacle, which upon emergence from said second opening of said elongated mixing receptacle is filtered off, dried and the surfactant removed.

### CROSS-REFERENCE TO RELATED APPLICATIONS

This application is a Continuation-in-Part of International Application No. PCT/EP2009/055122, filed Apr. 28, 2009, which claims the benefit of U.S. provisional application No. 61/201,532, filed Dec. 11, 2008, U.S. provisional patent application No. 61/137,673, filed Aug. 1, 2008, U.S. provisional application No. 61/125,795, filed Apr. 28, 2008, Great Britain patent application no. 09 033 95.2, filed Feb. 27, 2009, and Great Britain patent application no. 08 076 96.0, filed Apr. 28, 2008. This application also claims the benefit of U.S. Application 61/324,070, filed Apr. 14, 2010. Each of these applications is hereby incorporated by reference in its entirety.

### TECHNICAL FIELD OF THE INVENTION

The present invention relates to methods of self-assembling ordered mesoporous silica materials and 2D-hexagonal ordered mesoporous silica materials under mildly acidic or neutral pH conditions. Moreover the present invention relates to ordered mesoporous materials with a narrow (substantially uniform) mesopore size distribution, which are obtained by such methods.

### BACKGROUND OF THE INVENTION

Several types of ordered mesoporous silica materials were synthesized in the past using strongly acidic (pH<2) or basic (pH>9) reaction conditions. The use of surfactants and amphiphilic polymers as structure directing agents of ordered mesoporous silica materials is known in the art. Kresge et al. (Nature 1992, 359, 710-712) reported on the synthesis of MCM-41 materials showing hexagonal arrangements of tubular mesopores. MCM-41 synthesis is performed under basic conditions using cationic surfactants.

Zhao et al. (Science, 1998, 279, 548-552) reported the synthesis of SBA type materials under strongly acidic conditions. SBA-15 with uniform pores of 4.6 to 10 nm is synthesized. Conditions for avoiding the formation of silica gel or amorphous silica have been investigated in detail with various poly(alkylene oxide) triblock copolymers (e.g. PEO-PPO-PEO and the reverse PPO-PEO-PPO) and with TMOS as a source of silica. The article teaches that suitable conditions include (a) triblock copolymer concentrations between 0.5 and 6% by weight in the reaction mixture, (b) temperatures between 35 and 80° C. and (c) a pH below the isoelectric point of silica. In a publication by Zhao et al. (J. Am. Chem. Soc. 1998, 120, 6024-6036) the use of alkyl poly (ethylene oxide) oligomeric surfactants and poly (alkylene oxide) triblock co-polymers in strong acidic media has been reported for the synthesis of cubic and hexagonal mesoporous silica structures with pore sizes from 1.6 to 10 nm. Pore sizes from 1.6 to 3.1 nm were obtained with alkyl poly(ethylene oxide) oligomeric surfactants already at room temperature. Ordered mesoporous materials with pores from 3 to 10 nm were obtained with poly(alkylene oxide) triblock copolymers at temperatures from 35 to 80° C.

The prior art teaches that to obtain ordering of silica at the meso-scale (2 to 50 nm), it is mandatory to adjust the pH of the synthesis mixture below pH=2, being the isoelectric point of silica. Moreover, the quality of the ordering of mesoporous materials synthesized at pH=2 reported by Attard et al. (Nature 1995, 378, 366-368) and Weissenberger et al. (Ber. Bunsenges. Phys. Chem. 1997, 101, 1679-1682) was lower than in materials synthesized under more acidic conditions.

S. Su Kim et al. in 2001 in Journal of Physical Chemistry B, volume 105, pages 7663-7670, reported the assembly of MSU-H silica's using either a one-step or a two-step assembly process using sodium silicate as the silica source (27% SiO₂, 14% NaOH) and Pluronic P123 as the nonionic structure-directing triblock copolymer surfactant. In the one-step process, the mesostructure was formed at a fixed assembly temperature of 308, 318 or 333 K and the surfactant and an amount of acetic acid equivalent to the hydroxide content of the sodium silicate solution were mixed at ambient temperature and then added to the sodium silicate solution to form a reactive silica in the presence of the structure directing surfactant. This allowed for the assembly of the hexagonal framework under pH conditions where both the silica precursor and the surfactant were primarily nonionic molecular species (pH=ca. 6.5) outside the pH zone in which a sodium acetate/acetic acid mixture exerts a buffering action (see definition below). Heating of the synthesis mixture at 308 K was required to obtained a well ordered mesoporous material. Both surface area and pore volume increased with synthesis temperature, which shows that the material synthesized at the lowest temperature was less well structured and contained regions with less porosity.

An ordered mesoporous silica material synthesized at pH's greater than 2 and less than 9 is required with improved structural uniformity.

The manufacture of ordered mesoporous silicas using prior art synthesis procedures is relatively slow requiring up to 24 hours and is run as a batch procedure. A much faster and preferably a continuous production process is desirable as to render the production process more efficient and less cumbersome to conduct. The present invention provides such process that is fast and enables continuous production of mesoporous silicas.

### SUMMARY OF THE INVENTION

The present invention solves the problems of the related art that to manufacture materials with mesopore sizes of 4 to 30 nm, preferably 7 to 30 nm, particularly preferably 11 to 30 nm, and yet more preferably 15 to 30 nm, or 4 to 16 nm, or 6 to 16 nm, without the use or addition during the process of an aromatic hydrocarbon such as 1,2,4-trimethylbenzene, one has to use severe acidic condition (pH<2) or severe basic condition (pH>9) in a synthesis process and more particularly in the reaction mixture in the assembly of the ordered mesoporous silica material

The present invention also solves the problems of the related art of having to use severe acidic condition (pH<2) or severe basic condition (pH>9) in the reaction mixture to manufacture materials with substantially uniformly sized mesopores above 10 nm without the use or without having to add an aromatic hydrocarbon such as 1,2,4-trimethylbenzene to the reaction mixture.

Ordered mesoporous silica materials of the present invention with a substantially uniform pore size, also above 10 nm, are thus prepared with a self assembling reaction mixture at a mild pH condition between pH 2 and pH 8 that is free of an aromatic hydrocarbon such as 1,2,4-trimethylbenzene.

2D-hexagonal ordered mesoporous silica materials of the present invention with a substantially uniform pore size can thus be prepared with a self assembling reaction mixture at a mild pH condition between pH 2 and pH 8 that is free of an aromatic hydrocarbon such as 1,2,4-trimethylbenzene by the addition to such reaction mixture of a buffer with a pH greater than 2 and less than 8 even at room temperature if within the buffer zone of the acid component of the buffer.

Surprisingly, adding an aqueous solution of a poly(alkylene oxide) triblock copolymer with an acid with a pKa<2, an acid with a pKa in the range of 3 to 9 or a buffer to an aqueous alkaline silicate solution to give pH conditions from mildly acidic (pH>2) to mildly basic (pH<8) pH and allowing a reaction to take place between the components at the buffered pH and at a temperature in the range of 10 to 100° C. produced ordered mesoporous silica materials with substantially uniform pore size was obtained with substantially uniform pore size with a narrow mesopore size distribution around a maximum pore size selected from the size values of 5 nm, 7 nm, 9 nm, 11 nm, 13 nm, 15 nm, 17 nm, 19 nm, 21 nm, 23 nm, 25 nm, 27 nm or 29 nm, after filtering off, drying and calcinating the reaction product, even if the reaction had been carried out at room temperature. If the aqueous solution of poly(alkylene oxide) triblock copolymer with an acid with a pKa<2 was used, the additional presence of akali or alkaline earth hydroxide in the solution prior to addition to the aqueous alkaline silicate solution was found to have an adverse effect upon the assembly of an ordered mesoporous silica material. However, the additional presence of an organic cationic species such as tetraalkylammonium cation, such as tetramethyl ammonium or tetrapropylammonium, preferably tetrapropylammonium or a tetrapropylammonium generating molecule such as tetrapropylammonium hydroxide, in the aqueous solution of a poly(alkylene oxide) triblock copolymer with an acid with a pKa<2 had no adverse effect upon the production of an ordered mesoporous silica with substantially uniform pore size and was beneficial. The different effect of the presence of an alkali or alkaline earth hydroxide, such as calcium hydroxide with a pKa of 11.43, barium hydroxide with a pKa of 16.02, sodium hydroxide with a pKa of 13.8, potassium hydroxide with a pKa pf 13.5 and lithium hydroxide with a pKa of 14.36, in the aqueous solution of poly(alkylene oxide) triblock copolymer and an acid with a pKa of less than 2 than in the case of the further addition of tetraalkylammonium cations e.g. as a tetraalkylammonium hydroxide, a strong base with a pKa of 13.8, is surprising in view of the similar pKa's.

The COK-10 materials produced in the presence of an acid with pKa<2 and the COK-12 materials produced in the presence of an acid with a pKa in the range of 3 to 9 or a buffer have several advantages compared to ordered mesoporous materials known in the art of which some important advantages can be summarized as follows:
1. The synthesis avoids the use of very acidic conditions (such as in the procedures for the synthesis of SBA materials); or basic conditions (such as for the synthesis of MCM-41). The manufacturing is less demanding with respect to corrosion of synthesis vessels. There is no production of strongly acidic or basic waste streams.
2. The synthesis approaches known in the art typically lead to materials with mesopore sizes of 2 to 10 nm. The synthesis of pores wider than 10 nm is difficult and necessitates the use of swelling agents such as trimethylbenzene. According to the present invention, the use of mild pH conditions facilitates the formation of mesopores in the range 4 to 30 nm.
3. COK-10 materials with their wide mesopores are desirable for many applications, e.g. for the immediate release of poorly soluble drugs, for the preparation of HPLC columns, in biotechnology for supporting enzymes, proteins, nucleic acids or other types of biomolecules.

In accordance with the purpose of the invention, as embodied and broadly described herein, one embodiment of the invention is directed to a broadly drawn new process to manufacture new mesoporous materials of narrow mesopore size distribution (COK-10) under pH conditions in the self assembling reaction medium of which the pH selected from mildly acidic pH (pH>2) to mildly basic pH (pH<8). As compared to a MCM or a SBA framework mesoporous silica material which has been produced under more severe pH conditions in the reaction medium (pH>2 or pH<8) and these COK-10 materials if loaded with a poorly water soluble bioactive species into its pores have an improved releasing speed of these poorly water soluble bioactive species into a watery medium.

Aspects of the present invention are realized by a process for self-assembling an ordered mesoporous silica material with a substantially uniform pore size in the range of 4 to 30 nm, or 7 to 30 nm, or 4 to 14 nm, or 6 to 14 nm, comprising the steps of:

preparing an aqueous solution 1 comprising an aqueous alkali silicate solution;
preparing an aqueous solution 2, exclusive of an alkali or alkaline earth hydroxide e.g. an alkaline hydroxide such as sodium hydroxide, the aqueous solution 2 comprising a poly(alkylene oxide) triblock copolymer and an acid with a pKa of less than 2, preferably less than 1;
adding said aqueous solution 1 to said aqueous solution 2 giving a pH greater than 2 and less than 8 and allowing a reaction between the components to take place at a temperature in the range of 10 to 100° C., preferably 20 to 90° C., and filtering off, drying and calcinating the reaction product to produce said ordered mesoporous silica material with a substantially uniform pore size.

Aspects of the present invention are also realized by an ordered mesoporous silica material with a substantially uniform pore size in the range of 4 to 30 nm obtainable by above-mentioned process.

Aspects of the present invention are also realized by a pharmaceutical composition comprising the above-mentioned ordered mesoporous silica material and a bioactive species.

Aspects of the present invention are also realized by a process for self-assembling a 2D-hexagonal ordered mesoporous silica material with a substantially uniform pore size in the range of 4 to 12 nm comprising the steps of:

preparing an aqueous solution 1 comprising an alkali silicate solution;

preparing an aqueous solution 3 comprising a poly(alkylene oxide) triblock copolymer and a buffer with a pH greater than 2 and less than 8, said buffer having an acid and a base component;

adding said aqueous alkali silicate solution to said aqueous solution giving a pH greater than 2 and less than 8 and allowing a reaction between the components to take place at a temperature in the range of 10 to 100° C., preferably 20 to 90° C., and

filtering off, drying and calcinating the reaction product to produce said 2D-hexagonal ordered mesoporous silica material with a substantially uniform pore size.

Aspects of the present invention are also realized by a process for self-assembling a 2D-hexagonal ordered mesoporous silica material with a substantially uniform pore size in the range of 4 to 14 nm, or 6 to 14 nm, or 4 to 12 nm, or 6 to 12 nm, comprising the steps of:

preparing an aqueous solution 1 comprising an alkali silicate solution;

preparing an aqueous solution 4 comprising a poly(alkylene oxide) triblock copolymer and an acid with a pKa in the range 3 to 9;

adding said aqueous solution 1 to said aqueous solution 3 thereby realizing a pH greater than 2 and less than 8 which is within a range of 1.5 pH units above and 1.5 pH units below a pH having the same numerical value as a pKa of said acid with a pKa in the range of 3 to 9 and allowing a reaction between the components to take place at a temperature in the range of 10 to 100° C., and

filtering off, drying and calcinating the reaction product to produce said 2D-hexagonal ordered mesoporous silica material with a substantially uniform pore size.

Aspects of the present invention are also realized by a 2D-hexagonal ordered mesoporous silica material with a substantially uniform pore size in the range of 4 to 14 nm, or 6 to 14 nm, 4 to 12 nm, or 6 to 12 nm, obtainable by above-mentioned processes, with the ratio of Q3 to Q4 silica obtained using ²⁹Si MAS NMR preferably being less than 0.65 and particularly preferably less than 0.60.

Aspects of the present invention are also realized by a 2D-hexagonal ordered mesoporous silica material with a substantially uniform pore size in the range of 4 to 14 nm, or 6 to 14 nm, 4 to 12 nm, or 6 to 12 nm, with the ratio of Q3 to Q4 silica obtained using ²⁹Si MAS NMR preferably being less than 0.65 and particularly preferably less than 0.60.

Aspects of the present invention are also realized by a pharmaceutical composition comprising the above-mentioned 2D-hexagonal ordered mesoporous silica material and a bioactive species.

In addition, the present invention concerns a process which enables the ordered mesoporous silicas and 2D-hexagonal ordered mesoporous silica materials with substantially uniformly sized mesopores above 4 nm disclosed herein to be realized in a one step reaction of an aqueous silica precursor solution with an aqueous surfactant solution in a continuous process in which the self-assembled mesostructured precursor material is instantly produced (less than 10 s) without an ageing step, and in particular without an ageing step at isothermal elevated temperature conditions e.g. at 90° C., compared with the up to 24 hours disclosed in the prior art, while still enabling the synthesis to be realised without using severe acidic condition (pH<2) or severe basic condition (pH>9) in the reaction mixture.

In one embodiment, the invention concerns a process for producing under mildly acidic or neutral pH condition such ordered mesoporous silica materials and 2D-hexagonal ordered mesoporous silica materials or their mesostructured precursor materials whereby an elongated mixing receptacle (conduit) receives a first aqueous solution of the surfactant from a first reservoir member and a second aqueous solution of the silica precursor from a second reservoir member and both are fed or delivered to the elongated mixing receptacle (conduit, tube) and the self-assembled ordered mesoporous silica material emerges from the elongated mixing receptacle even in the case of residence times of less than 10 s achieved with linear velocities of the aqueous solution of the surfactant from a first reservoir member and of the second aqueous solution of the silica precursor in the range of 1000 to 3000 m/h.

Synthesis experiments at pHs of 4.8, 5.0 and 6.0 yielded ordered porous materials at room temperature, with the characteristics given in Table 1 below, surprisingly showing that addition of sodium silicate solution to the buffered surfactant leads to a quasi immediate silica polymerisation. TEM micrographs show that materials recovered after 1 minute were ordered as shown in FIGS. 58A and 58B.

TABLE 1

| | synthesis | Buffer | | | | Wall | |
|---|---|---|---|---|---|---|---|
| | time | strength | | d₁₀ | dₚ | thickness* | Vₚ |
| pH | [min] | [M] | Na:Si | [nm] | [nm] | [nm] | [cm³/g] |
| 4.8 | 1 | 0.17 | 1.13 | — | 4.3 | ca. 4.0 | 0.36 |
| 5.0 | 1440 | 0.17 | 1.13 | 8.7 | 5.8 | 4.3 | 0.45 |
| 6.0 | 1440 | 0.26 | 2.31 | 8.4 | 6.3 | 3.5 | 0.50 |

*calculated from the combination of d₁₀ spacing and pore size

Moreover, this is achieved in the absence of an aromatic hydrocarbon such as 1,2,4-trimethylbenzene in the reaction mixture.

Surprisingly, highly ordered mesoporous COK-12 silicas with substantially uniformly sized mesopores above 2 nm, e.g. in the range of 4 to 30 nm, or 7 to 30 nm, or 4 to 14 nm, or 6 to 14 nm, can be produced at a pH between 2 and 8, preferably 5-6, in a continuous synthesis process by mixing a jet of aqueous sodium silicate solution (water glass) with a jet of a buffered aqueous solution of the block copolymer P123 surfactant in a tube at room temperature. Upon contact of the jets silica condensation and particle formation is readily visible. The silica particles are immediately collected and separated and exhibit a hexagonal ordering at the mesoscale and mesopores of uniform size. Highly ordered mesoporous COK-12 silicas are thereby produced in times as low as a minute with full condensation in less than 5 minutes compared with upon to 24 hours using conventional templating processes.

Aspects of the present invention are realized by a process for producing an ordered mesoporous silica material with a substantially uniform pore size using a self-assembling method, the process comprising the steps of:

preparing an aqueous solution A comprising a silica precursor;
preparing an aqueous solution B, exclusive of an alkali or alkaline earth hydroxide, said aqueous solution B comprising a poly(alkylene oxide) triblock copolymer and an acid with a pKa in the range 3 to 9;
feeding said solution A and said solution B as liquid streams into an elongated mixing receptacle having a first and a second opening such that the liquid streams of solution A and solution B are each discharged independently into said first opening of said elongated mixing receptacle such that they directly impinge thereby giving a pH in the resulting mixture of greater than 2 and less than 8 and producing a reaction product at a temperature in the range of 10 to 100° C. in said elongated mixing receptacle,
which upon emergence from said second opening of said elongated mixing receptacle is filtered off, dried and surfactant removal to produce said ordered mesoporous silica material with a substantially uniform pore size;
and wherein said process is exclusive of an ageing step and said liquid streams of solution A and solution B are each discharged independently into said first opening of said elongated mixing receptacle each independently optionally at a linear velocity greater than 5 m/h.

Aspects of the present invention are also realized by a process for producing an ordered mesoporous silica material with a substantially uniform pore size using a self-assembling method, the process comprising the steps of:

preparing an aqueous solution A comprising a silica precursor, for example an alkali silicate, silicic acids or a tetraalkyl orthosilicate such as tetraethyl orthosilicate (TEOS), tetramethyl orthosilicate (TMOS) and tetrapropyl orthosilicate (TPOS);
preparing an aqueous solution B, exclusive of an alkali or alkaline earth hydroxide, said aqueous solution B comprising a poly(alkylene oxide) triblock copolymer, an acid with a pKa in the range 3 to 9 and optionally an alkali salt of an acid with a pKa in the range 3 to 9;
feeding said solution A and said solution B as liquid streams into an elongated mixing receptacle having a first and a second opening such that the liquid streams of solution A and solution B are each discharged independently into said first opening of said elongated mixing receptacle each independently at a linear velocity greater than 5 m/h such that they directly impinge thereby giving a pH in the resulting mixture of greater than 2 and less than 8 and producing a reaction product at a temperature in the range of 10 to 100° C. in said elongated mixing receptacle
which upon emergence from the second opening of the elongated mixing receptacle is filtered off, dried and surfactant removal to produce said ordered mesoporous silica material with a substantially uniform pore size; and wherein the process is optionally exclusive of an ageing step.

Aspects of the present invention are realized by a process for producing an ordered mesoporous silica material with a substantially uniform pore size using a self-assembling method, the process comprising the steps of:

preparing an aqueous solution A comprising a silica precursor;
preparing an aqueous solution B, exclusive of an alkali or alkaline earth hydroxide, said aqueous solution B comprising a poly(alkylene oxide) triblock copolymer and an acid with a pKa in the range 3 to 9;
feeding said solution A and said solution B as liquid streams into an elongated mixing receptacle having a first and a second opening such that the liquid streams of solution A and solution B are each discharged independently into said first opening of said elongated mixing receptacle such that they directly impinge thereby giving a pH in the resulting mixture of greater than 2 and less than 8 and producing a reaction product at a temperature in the range of 10 to 100° C. in said elongated mixing receptacle
which upon emergence from said second opening of said elongated mixing receptacle is filtered off, dried and surfactant removal to produce said ordered mesoporous silica material with a substantially uniform pore size;
and wherein said process is optionally exclusive of an ageing step and said liquid streams of solution A and solution B are each discharged independently into said first opening of said elongated mixing receptacle each independently optionally at a linear velocity greater than 5 m/h.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1: illustrates an X-ray scattering pattern of as-synthesized COK-10 material of Example 1, recorded at the BM26B beamline of the European Synchrotron radiation facility (ESRF) in transmission geometry.

FIG. 2: Top: provides a nitrogen adsorption isotherm of calcined COK-10 material of Example 1. Bottom: BJH mesopore size distribution calculated from desorption branch.

FIG. 3: provides SEM images of calcined COK-10 material of Example 1 at two magnifications. Samples were coated with gold. Images were obtained with a Philips (FEI) SEM XL30 FEG.

FIG. 4: provides X-ray scattering pattern of as-synthesized material of Example 2, recorded at the BM26B beamline of the European Synchrotron radiation facility (ESRF) in transmission geometry.

FIG. 5: Top: provides a nitrogen adsorption isotherm of calcined COK-10 material of Example 2. Bottom: BJH mesopore size distribution calculated from desorption branch.

FIG. 6: illustrates SEM images of calcined COK-10 material of Example 2 at two magnifications. Samples were coated with gold. Images were obtained with a Philips (FEI) SEM XL30 FEG.

FIG. 7: provides a X-ray scattering pattern of as-synthesized material of Example 3, recorded at the BM26B beamline of the European Synchrotron radiation facility (ESRF) in transmission geometry.

FIG. 8: displays SEM images of calcined material of Example 3 at two magnifications. Samples were coated with gold. Images were obtained with a Philips (FEI) SEM XL30 FEG.

FIG. 9: provides the Nitrogen adsorption isotherm of the material synthesized in Example 3 (top) and mesopore size distribution according to the BJH model (bottom).

FIG. 10: Top: provides a nitrogen adsorption isotherm of calcined SBA-15 material of Example 4. Bottom: BJH mesopore size distribution calculated from the desorption branch of the isotherm.

FIG. 11: displays SEM images of calcined SBA-15 material of Example 4 at two magnifications. Samples were coated with gold. Images were obtained with a Philips (FBI) SEM XL30 FEG instrument.

FIG. 12: Top: provides a nitrogen adsorption isotherm of calcined COK-10 material of example 7. Bottom: BJH pore size distribution calculated from desorption branch.

FIG. 13: displays a SEM image of calcined COK-10 material of example 7. The sample was coated with gold. Images were obtained with a Philips (FEI) SEM XL30 FEG instrument.

FIG. 14: illustrates an X-ray scattering pattern of calcined COK-10 material of Example 7, recorded at the BM26B beamline of the European Synchrotron radiation facility (ESRF) in transmission geometry.

FIG. 15: is a graphic display of in vitro release of itraconazole from COK-10 sample of experiment 1. Release medium: Simulated gastric fluid with 0.05 wt.-% SLS.

FIG. 16: is a graphic display of in vitro release of itraconazole from mesoporous material not according to the invention prepared in Experiment 3. Release medium: Simulated gastric fluid with 0.05 wt.-% SLS.

FIG. 17: is a graphic display of in vitro release of itraconazole from SBA-15 synthesized in comparative Example 4. Release medium: Simulated gastric fluid with 0.05 wt.-% SLS.

FIG. 18: provides Top: nitrogen adsorption (right curve) and desorption isotherm (left curve) of calcined COK-10 material of Example 11. Bottom: BJH pore size distribution calculated from adsorption branch. Measurement was performed on a Micromeritics Tristar apparatus. Prior to measurement, the sample was pretreated at 300° C. for 10 h (ramp: 5° C./min).

FIG. 19: is a SEM image of calcined COK-10 material of Example 11. Samples were coated with gold. Images were obtained with a Philips (FEI) SEM XL30 FEG.

FIG. 20: demonstrates an X-ray scattering pattern of calcined COK-10 material of Example 11, recorded at the BM26B beamline of the European Synchrotron radiation facility (ESRF) in transmission geometry.

FIG. 21: provides Top: nitrogen adsorption (right curve) and desorption isotherm (left curve) of calcined COK-10 material of Example 12. Bottom: BJH pore size distribution calculated from adsorption branch. Measurement was performed on a Micromeritics Tristar apparatus. Prior to measurement, the sample was pretreated at 300° C. for 10 h (ramp: 5° C./min).

FIG. 22: illustrates an X-ray scattering pattern of calcined COK-10 material of Example 12, recorded at the BM26B beamline of the European Synchrotron radiation facility (ESRF) in transmission geometry.

FIG. 23: provides Top: nitrogen adsorption (right curve) and desorption isotherm (left curve) of calcined COK-10 material of Example 13. Bottom: BJH pore size distribution calculated from adsorption branch. Measurement was performed on a Micromeritics Tristar apparatus. Prior to measurement, the sample was pretreated at 300° C. for 10 h (ramp: 5° C./min).

FIG. 24: illustrates an X-ray scattering pattern of calcined COK-10 material of Example 13, recorded at the BM26B beamline of the European Synchrotron radiation facility (ESRF) in transmission geometry.

FIG. 25: X-ray scattering pattern of as-synthesized (thin line) and the calcined (thick line) COK-12 material of Example 14, recorded at the BM26B beamline of the European Synchrotron radiation facility (ESRF) in transmission geometry.

FIG. 26: Top: nitrogen adsorption isotherm of calcined COK-12 material of Example 14. Bottom: BJH mesopore size distribution calculated from desorption branch. Measurement was performed on a Micromeritics Tristar 3000 apparatus. Prior to measurement, the sample was pretreated at 300° C. for 10 h (ramp: 5° C./min).

FIG. 27: SEM images of calcined COK-12 material of Example 14 at two magnifications. Samples were coated with gold. Images were obtained with a Philips (FEI) SEM XL30 FEG.

FIG. 28: X-ray scattering pattern of calcined (thick line) COK-12 material of Example 15, recorded at the BM26B beamline of the European Synchrotron radiation facility (ESRF) in transmission geometry.

FIG. 29: Top: nitrogen adsorption isotherm of calcined COK-12 material of Example 15. Bottom: BJH mesopore size distribution calculated from desorption branch. Measurement was performed on a Micromeritics Tristar 3000 apparatus. Prior to measurement, the sample was pretreated at 200° C. for 10 h (ramp: 5° C./min).

FIG. 30: SEM images of calcined COK-12 material of Example 15 at two magnifications. Samples were coated with gold. Images were obtained with a Philips (FEI) SEM XL30 FEG.

FIG. 31: X-ray scattering pattern of as-synthesized (thin line) and the calcined (thick line) COK-12 material of Example 16, recorded at the BM26B beamline of the European Synchrotron radiation facility (ESRF) in transmission geometry.

FIG. 32: Top: nitrogen adsorption isotherm of calcined COK-12 material of Example 16. Bottom: BJH mesopore size distribution calculated from desorption branch. Measurement was performed on a Micromeritics Tristar 3000 apparatus. Prior to measurement, the sample was pretreated at 300° C. for 10 h (ramp: 5° C./min).

FIG. 33: SEM images of calcined COK-12 material of Example 16 at two magnifications. Samples were coated with gold. Images were obtained with a Philips (FEI) SEM XL30 FEG.

FIG. 34: X-ray scattering pattern of calcined (thick line) COK-12 material of Example 17, recorded at the BM26B beamline of the European Synchrotron radiation facility (ESRF) in transmission geometry.

FIG. 35: Top: nitrogen adsorption isotherm of calcined COK-12 material of Example 17. Bottom: BJH mesopore size distribution calculated from desorption branch. Measurement was performed on a Micromeritics Tristar 3000 apparatus. Prior to measurement, the sample was pretreated at 200° C. for 10 h (ramp: 5° C./min).

FIG. 36: Top: nitrogen adsorption isotherm of calcined COK-12 material of Example 18. Bottom: BJH mesopore size distribution calculated from desorption branch. Measurement was performed on a Micromeritics Tristar 3000 apparatus. Prior to measurement, the sample was pretreated at 200° C. for 10 h (ramp: 5° C./min).

FIG. 37: X-ray scattering pattern of as-synthesized (thin line) and the calcined (thick line) COK-12 material of Example 19, recorded at the BM26B beamline of the European Synchrotron radiation facility (ESRF) in transmission geometry.

FIG. 38: Top: nitrogen adsorption isotherm of calcined COK-12 material of Example 19. Bottom: BJH mesopore size distribution calculated from desorption branch. Measurement was performed on a Micromeritics Tristar 3000 apparatus. Prior to measurement, the sample was pretreated at 200° C. for 10 h (ramp: 5° C./min).

FIG. 39: X-ray scattering pattern of as-synthesized (thin line) and the calcined (thick line) COK-12 material of Example 20, recorded at the BM26B beamline of the European Synchrotron radiation facility (ESRF) in transmission geometry.

FIG. 40: Top: nitrogen adsorption isotherm of calcined COK-12 material of Example 20. Bottom: BJH mesopore size distribution calculated from desorption branch. Measurement was performed on a Micromeritics Tristar 3000 apparatus. Prior to measurement, the sample was pretreated at 200° C. for 10 h (ramp: 5° C./min).

FIG. 41: X-ray scattering pattern of as-synthesized (thin line) and the calcined (thick line) COK-12 material of Example 21, recorded at the BM26B beam line of the European Synchrotron radiation facility (ESRF) in transmission geometry.

FIG. 42: Top: nitrogen adsorption isotherm of calcined COK-12 material of Example 21. Bottom: BJH mesopore size distribution calculated from desorption branch. Measurement was performed on a Micromeritics Tristar 3000 apparatus. Prior to measurement, the sample was pretreated at 200° C. for 10 h (ramp: 5° C./min).

FIG. 43: SEM images of calcined COK-12 material of Example 21 at two magnifications. Samples were coated with gold. Images were obtained with a Philips (FEI) SEM XL30 FEG.

FIG. 44: X-ray scattering pattern of as-synthesized (thin line) and the calcined (thick line) COK-12 material of Example 22, recorded at the BM26B beamline of the European Synchrotron radiation facility (ESRF) in transmission geometry.

FIG. 45: Top: nitrogen adsorption isotherm of calcined COK-12 material of Example 22. Bottom: BJH mesopore size distribution calculated from desorption branch. Measurement was performed on a Micromeritics Tristar 3000 apparatus. Prior to measurement, the sample was pretreated at 300° C. for 10 h (ramp: 5° C./min).

FIG. 46: SEM images of calcined COK-12 material of Example 22 at two magnifications. Samples were coated with gold. Images were obtained with a Philips (FEI) SEM XL30 FEG.

FIG. 47: Top: nitrogen adsorption isotherm of calcined COK-12 material of Example 23. Bottom: BJH mesopore size distribution calculated from desorption branch. Measurement was performed on a Micromeritics Tristar 3000 apparatus. Prior to measurement, the sample was pretreated at 200° C. for 10 h (ramp: 5° C./min).

FIG. 48: Top: nitrogen adsorption isotherm of calcined COK-12 material of Example 24. Bottom: BJH mesopore size distribution calculated from desorption branch. Measurement was performed on a Micromeritics Tristar 3000 apparatus. Prior to measurement, the sample was pretreated at 200° C. for 10 h (ramp: 5° C./min).

FIG. 49: Scheme of experimental setup in which A is the aqueous solution A; AT is the tube transporting the aqueous solution A; B is the aqueous solution B; BT is the tube transporting the aqueous solution B; M is the mixing tube; C is the collector; S is the separator; and D is the drier/calcinating oven.

FIG. 50A: nitrogen adsorption of COK-12 calcined mesoporous silica material of Example 25 (squares) and desorption isotherm (filled diamonds) of calcined material.

FIG. 50B: BJH pore size distribution calculated from adsorption branch.

FIGS. 51A, 51B and 51C: SEM micrographs of COK-12 calcined mesoporous silica material of Example 25. Samples were coated with gold. Images were obtained with a Philips (FEI) SEM XL30 FEG.

FIG. 52A: nitrogen adsorption of COK-12 calcined mesoporous silica material of Example 26 (squares) and desorption isotherm (filled diamonds) of calcined material.

FIG. 52B: BJH pore size distribution calculated from adsorption branch.

FIG. 53A: nitrogen adsorption of COK-12 calcined mesoporous silica material of Example 27 (filled diamonds) and desorption isotherm (squares) of calcined material.

FIG. 53B: BJH pore size distribution calculated from adsorption branch.

FIGS. 54A and 54B: SEM micrographs of COK-12 calcined mesoporous silica material of Example 27. Samples were coated with gold. Images were obtained with a Philips (FEI) SEM XL30 FEG.

FIG. 55A: nitrogen adsorption of COK-12 calcined mesoporous silica material of Example 28 (filled diamonds) and desorption isotherm (squares) of calcined material.

FIG. 55B: BJH pore size distribution calculated from adsorption branch.

FIGS. 56A, 56B and 56C: TEM micrographs of COK-12 calcined mesoporous silica material of Example 28. Images were obtained with a Philips (FEI) SEM XL30 FEG,

FIG. 57: nitrogen absorption of COK-12 calcined mesoporous silica material of Example 29 (diamonds) and desorption isotherm (filled squares) of calcined material.

FIGS. 58A and 58B: TEM micrographs of COK-12 calcined silica material of Example 29.

### DETAILED DESCRIPTION OF THE INVENTION

The following detailed description of the invention refers to the accompanying drawings. The same reference numbers in different drawings identify the same or similar elements. Also, the following detailed description does not limit the invention. Instead, the scope of the invention is defined by the appended claims and equivalents thereof.

Several documents are cited throughout the text of this specification. Each of the documents herein (including any manufacturer's specifications, instructions etc.) are hereby incorporated by reference; however, there is no admission that any document cited is indeed prior art of the present invention.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it doe not exclude other elements or steps. It is thus to be interpreted as specifying the presence of the stated features, integers, steps or components as referred to, but doe not preclude the presence or addition of one or more other features, integers, steps or components, or groups thereof. Thus, the scope of the expression “a device comprising means A and B” should not be limited to the devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

The following terms are provided solely to aid in the understanding of the invention.

### DEFINITIONS

The terms mesoscale, mesopore, mesoporous and the like, as used in this specification, may refer to structures having feature sizes in the range of 5 nm to 100 nm, in particular in the range of 2 nm to 50 nm. No particular spatial organization or method of manufacture is implied by the term mesoscale as used here. Hence, a mesoporous material includes pores, which may be ordered or randomly distributed, having a diameter in the range of 5 nm to 100 nm, whereas a nanoporous material includes pores having a diameter in the range of 0.5 nm to 1000 nm.

According to IUPAC notation [see J. Rouquerol et al., Pure & Appl. Chem., 66 (1994) 1739-1758), microporous materials have pore diameters of less than 2 nm, mesoporous materials have pore diameters between 2 nm and 50 nm and macroporous materials have pore diameters of greater than 50 nm. A nanoporous material includes pores having a diameter in the range of 0.3 nm to 100 nm.

The terms narrow pore size distribution and substantially uniform pore size, as used in disclosing the present application, means a pore size distribution curve showing the derivative of pore volume (dV) as a function of pore diameter such that at a point in the curve that is half the height thereof, the ratio of the width of the curve (the difference between the maximum pore diameter and the minimum pore diameter at the half height) to the pore diameter at the maximum height of the plot (as hereinabove described) is no greater than 0.75. The pore size distribution of materials prepared by the present invention may be determined by nitrogen adsorption and desorption and producing from the acquired data a plot of the derivative of pore volume as a function of pore diameter. The nitrogen adsorption and desorption data may be obtained by using instruments available in the art (for example Micrometrics ASAP 2010) which instruments are also capable of producing a plot of the derivative of pore volume as a function of the pore diameter. In the micro pore range, such a plot may be generated by using the slit pore geometry of the Horvath-Kawazoe model, as described in G. Horvath, K. Kawazoe, J. Chem. Eng. Japan, 16(6), (1983), 470. In the mesopore range, such plot may be generated by the methodology described in E. P. Barrett, L. S. Joyner and P. P. Halenda, J. Am. Chem. Soc., 73 (1951), 373-380.

The term “aqueous” as used in disclosing the present invention means relating to or made with water.

The term “elongated mixing receptacle”, as used in disclosing the present invention, means any form of receptable with two openings e.g. a tube, a pipe, a conduit etc.

The term “silica precursor”, as used in the disclosing the present invention, means any compound from which silica can be produced in the process of the present invention which includes alkali silicates, silicic acids and tetraalkyl orthosilicates e.g. tetraethyl orthosilicate (TEOS), tetramethyl orthosilicate (TMOS) and tetrapropyl orthosilicate (TPOS).

The term “surfactant removal”, as used in disclosing the present invention, means a process step in which surfactant is removed, such processes including calcination e.g. at 200° C. and extraction e.g. with super-cooled carbon dioxide or ethanol.

The term “ageing step”, as used in disclosing the present invention, means any step in which the product of the reaction of solutions A and B has to be further treated to complete the process of producing an ordered mesoporous silica material with a substantially uniform pore size e.g. by holding it under isothermal conditions for a period of time.

The term “practically insoluble” as used herein applies to drugs that are essentially totally water-insoluble or are at least poorly water-soluble. More specifically, the term is applied to any drug that has a dose (mg) to aqueous solubility (mg/ml) ratio greater than 100 ml, where the drug solubility is that of the neutral (for example, free base or free acid) form in unbuffered water. This meaning is to include, but is not to be limited to, drugs that have essentially no aqueous solubility (less than 1.0 mg/ml).

Based on the BCS, “poorly water-soluble” can be defined as compounds whose highest dose is not soluble in 250 mL or less of aqueous media from pH 1.2 to 7.5 at 37° C. See Cynthia K. Brown, et al., “Acceptable Analytical Practices for Dissolution Testing of Poorly Soluble Compounds”, Pharmaceutical Technology (December 2004).

According to the manual, Pharmaceutics (M. E. Aulton) for any solvent solubility is defined as the amount of a solvent (g) required to solve 1 g op the compounds whereby the following solubility qualification are defined: 10-30 g (soluble); 30-100 g (“sparingly soluble”); 100-1000 g (“slightly soluble”); 1000-10000 g (“very slightly soluble” or “poorly soluble”) and more than 10000 (practically insoluble).

The terms “drug” and “bioactive compound” will be widely understood and denotes a compound having beneficial prophylactic and/or therapeutic properties when administered to, for example, humans. Further, the term “drug per se” is used throughout this specification for the purposes of comparison, and means the drug when in an aqueous solution/suspension without the addition of any excipients.

The term “antibody” refers to intact molecules as well as fragments thereof, which are capable of binding to the epitope determinant of the relevant factor or domain of the factor. An “Fv” fragment is the smallest antibody fragment, and contains a complete antigen recognition site and a binding site. This region is a dimer (VH-VL dimer) wherein the variable regions of each of the heavy chain and light chain are strongly connected by a non-covalent bond. The three CDRs of each of the variable regions interact with each other to form an antigen-binding site on the surface of the VH-VL dimer. In other words, a total of six CDRs from the heavy and light chains function together as an antibody's antigen-binding site. However, a variable region (or a half Fv, which contains only three antigen-specific CDRs) alone is also known to be able to recognize and bind to an antigen, although its affinity is lower than the affinity of the entire binding site. Thus, a preferred antibody fragment of the present invention is an Fv fragment, but is not limited thereto. Such an antibody fragment may be a polypeptide which comprises an antibody fragment of heavy or light chain CDRs which are conserved, and which can recognize and bind its antigen. A Fab fragment (also referred to as F(ab)) also contains a light chain constant region and heavy chain constant region (CH1). For example, papain digestion of an antibody produces the two kinds of fragments: an antigen-binding fragment, called a Fab fragment, containing the variable regions of a heavy chain and light chain, which serve as a single antigen-binding domain; and the remaining portion, which is called an “Fc” because it is readily crystallized. A Fab′ fragment is different from a Fab fragment in that a Fab′ fragment also has several residues derived from the carboxyl terminus of a heavy chain CH1 region, which contains one or more cysteine residues from the hinge region of an antibody. A Fab′ fragment is, however, structurally equivalent to Fab in that both are antigen-binding fragments which comprise the variable regions of a heavy chain and light chain, which serve as a single antigen-binding domain. Herein, an antigen-binding fragment comprising the variable regions of a heavy chain and light chain which serve as a single antigen-binding domain, and which is equivalent to that obtained by papain digestion, is referred to as a “Fab-like antibody”, even when it is not identical to an antibody fragment produced by protease digestion. Fab′-SH is Fab′ with one or more cysteine residues having free thiol groups in its constant region.

The term bioactive species, as used in disclosing the present invention, means drugs and antibodies.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermo-dynamics, such a solid dispersion will be called “a solid solution” hereinafter. Solid solutions are preferred physical systems because the components therein are usually readily bioavailable to the organisms to which they are administered. This advantage can probably be explained by the ease with which said solid solutions can form liquid solutions when contacted with a liquid medium such as gastric juice. The ease of dissolution may be attributed at least in part to the fact that the energy required for dissolution of the components from a solid solution is less than that required for the dissolution of components from a crystalline or microcrystalline solid phase.

The term “a solid dispersion” also comprises dispersions which are less homogenous throughout than solid solutions. Such dispersions are not chemically and physically uniform throughout or comprise more than one phase. For example, the term “a solid dispersion” also relates to particles having domains or small regions wherein amorphous, microcrystalline or crystalline (a), or amorphous, microcrystalline or crystalline (b), or both, are dispersed more or less evenly in another phase comprising (b), or (a), or a solid solution comprising (a) and (b). Said domains are regions within the particles distinctively marked by some physical feature, small in size compared to the size of the particle as a whole, and evenly and randomly distributed throughout the particle.

The term “room temperature” as used in this application means a temperature between 12-30° C., preferably between 18 and 28° C., more preferably between 19 and 27° C. and most preferably it is taken to be roughly between 20 and 26° C.

The term “low temperature” as used in this application means a temperature between 15 and 40° C., preferably between 18 and 23° C., more preferably between 20 and 30° C. and most preferably it is taken to be roughly between 22 and 28° C.

The term buffer zone of a buffer, as used in disclosing the present invention, means a zone of pH in the range of about 1.5 pH units above and about 1.5 pH units below the pH numerically equal to the pKa of the acid component of the buffer.

### Process for Self-Assembling an Ordered Mesoporous Silica Material with a Substantially Uniform Pore Size

Aspects of the present invention are realized by a process for self-assembling an ordered mesoporous silica material with a substantially uniform pore size in the range of 4 to 30 nm, or 7 to 30 nm, or 4 to 14 nm, or 6 to 14 nm, comprising the steps of: preparing an aqueous solution 1 comprising an aqueous alkali silicate solution; preparing an aqueous solution 2, exclusive of an alkali or alkaline earth hydroxide e.g. an alkaline hydroxide such as sodium hydroxide, the aqueous solution 2 comprising a poly(alkylene oxide) triblock copolymer; and an acid with a pKa of less than 2, preferably less than 1; adding said aqueous solution 1 to said aqueous solution 2 giving a pH greater than 2 and less than 8 i.e. above the isoelectric point of silica of 2; and allowing a reaction between the components to take place at a temperature in the range of 10 to 100° C., and filtering off, drying and calcinating the reaction product to produce said ordered mesoporous silica material with a substantially uniform pore size.

According to a preferred embodiment of the process for self-assembling an ordered mesoporous silica material with a substantially uniform pore size, according to the present invention, the aqueous solution 2 further comprises a tetraalkylammonium surfactant, preferably tetrapropylammonium hydroxide which generates a tetrapropylammonium cation or tetramethyl ammonium hydroxide which generates a tetramethylammonium cation. The presence of a tetraalkylammonium surfactant brings about changes in the ordered mesoporous silica produced.

The acid is largely removed during the washing process associated with the filtration process with any acid left being removed in the calcining process.

Variation in the reaction mixture pH within the ranges of present invention can together with reaction time or reaction temperature be used a condition to fine tune the pore size of the final ordered mesoporous silica material. The pore size increases slightly with increasing pH. The pore size increases more strongly with reaction temperature, but without substantially affecting the total pore volume. The pH at which the reaction is performed is preferably in the range of 2.2 to 7.8, particularly preferably in the range of 2.4 to 7.6, especially preferably in the range of 2.6 to 7.4.

In another embodiment the pH at which the reaction is carried out is preferably in the range of 2.8 to 7.2, particularly preferably in the range of 3 to 7.2, especially preferably in the range of 4 to 7 and particularly especially preferably in the range of 5 to 6.5.

In the process for self-assembling an ordered mesoporous silica material with a substantially uniform pore size, according to the present invention, the stirring speed is preferably in the range of 100 to 700 rpm.

Moreover it has been demonstrated that COK-10 materials can be produced in reaction mixtures with a pH greater than 2 and less than 8 under room temperature conditions (26° C. Example 11) or under low temperature conditions.

The process condition can be tuned to achieve ordered mesoporous silica materials with pore sizes selected from the range 4 to 30 nm, preferably selected from the range 7 to 30 nm, particularly preferably selected from a range 10 to 30 nm, yet more preferably selected from a range 10 to 30 nm.

The aqueous solution 1 is preferably an aqueous sodium silicate solution with at least 10% by weight of sodium hydroxide and at least 27% by weight of silica.

It will be apparent to those skilled in the art that various modifications and variations can be made in the amount of reagents or of intermediate such as the amphiphilic polymers where under Pluronic P123, or such as the tetraalkylammonium cation, in particular the tetrapropylammonium hydroxide or in the conditions of temperature, mixing speed or reaction time of the process of present invention and in construction of the system and method without departing from the scope or spirit of the invention. Such variations can be fined tuned to manufacture the narrow pore size distribution mesoporous materials of present invention with a desired maximum pore size within the range of 7 to 30 nm.

If an alkali silicate is used in solution A, an acid with a pKa in the range of 3 to 9 in solution B is sufficient to produce a pH upon mixing solutions A and B of greater than 2 and less than 8 without an alkali salt of an acid with a pKa in the range 3 to 9 being necessary. However, with alternative silica precursors in solution A, an acid with a pKa in the range of 3 to 9 in solution B may be insufficient to produce a pH upon mixing solutions A and B of greater than 2 and less than 8 without the addition of an alkali salt of an acid with a pKa in the range 3 to 9. The acid in the pKa range 3 to 9 may be present in a buffer with a pH in the range of 3 to 8 upon mixing solutions A and B or solution B may itself contain a buffer.

A particular embodiment of the ultrafast (1 to 100 s) procedure in accordance with this invention concerns a process for producing an ordered mesoporous silica material with a substantially uniform pore size using a self-assembling method, the process comprising the steps of: preparing an aqueous solution A, whereby solution A comprises a silica precursor; preparing an aqueous solution B, exclusive of an alkali or alkaline earth hydroxide, said aqueous solution B comprising a poly(alkylene oxide) triblock copolymer, an acid with a pKa in the range 3 to 9 and optionally an alkali salt of an acid with a pKa in the range 3 to 9; feeding said aqueous solution A and said solution B as liquid streams into an elongated mixing receptacle (conduit, tube) having a first opening and a second opening such that the liquid streams of aqueous solution A and aqueous solution B are each discharged independently into the first opening of said elongated mixing receptacle each independently at a linear velocity in the range of 10 m/h to 1000 m/h (a linear velocity greater than 30 m/h being preferred, a linear velocity greater than 100 m/h being particularly preferred and a linear velocity greater than 500 m/h being especially preferred) such that they directly impinge giving a pH in the resulting mixture of greater than 2 and less than 8 and producing a reaction product at a temperature in the range of 10 to 100° C. in said elongated mixing receptacle which upon emergence from the second opening is filtered off, dried and surfactant removal to produce said ordered mesoporous silica material with a substantially uniform pore size with a preferred residence time in the elongated mixing receptacle of 1 to 100 seconds; and wherein the process is optionally exclusive of an ageing step.

A particular embodiment of the instant (1 to 10 s) reaction of the present invention is a process for producing an ordered mesoporous silica material with a substantially uniform pore size using a self-assembling method, the process comprising the steps of: preparing an aqueous solution A, whereby solution A comprises a silica precursor; preparing an aqueous solution B, exclusive of an alkali or alkaline earth hydroxide, said aqueous solution B comprising a poly(alkylene oxide) triblock copolymer, an acid with a pKa in the range 3 to 9 and optionally an alkali salt of an acid with a pKa in the range 3 to 9; feeding said aqueous solution A and said solution B as liquid streams into an elongated mixing receptacle (conduit, tube) having a first opening and a second opening such that the liquid streams of aqueous solution A and aqueous solution B are each discharged independently into the first opening of said elongated mixing receptacle each independently at a linear velocity in the range of 1000 m/h to 3000 m/h such that they directly impinge giving a pH in the resulting mixture of greater than 2 and less than 8 and producing a reaction product at a temperature in the range of 10 to 100° C. in said elongated mixing receptacle which upon emergence from the second opening is filtered off, dried and surfactant removal to produce said ordered mesoporous silica material with a substantially uniform pore size with a preferred residence time in the elongated mixing receptacle of 1 to 10 seconds; and wherein the process is optionally exclusive of an ageing step.

According to a preferred embodiment of the process for producing an ordered mesoporous silica material, according to the present invention, said acid in the pKa range 3 to 9 is present in a buffer with a pH in the range of 3 to 8.

According to a preferred embodiment of the process for producing an ordered mesoporous silica material with a substantially uniform pore size, according to the present invention, the aqueous solution 2 further comprises a tetraalkylammonium surfactant, preferably tetrapropylammonium hydroxide which generates a tetrapropylammonium cation or tetramethyl ammonium hydroxide which generates a tetramethylammonium cation. The presence of a tetraalkylammonium surfactant brings about changes in the ordered mesoporous silica produced.

According to a further preferred embodiment of the present invention, said liquid streams of aqueous solution A and B are each discharged independently into said elongated mixing receptacle at a linear velocity greater than 10 m/h, with a linear velocity greater than 30 m/h being preferred, a linear velocity of 100 m/h being particularly preferred and a linear velocity greater than 1000 m/h being especially preferred.

According to a further preferred embodiment of the present invention, said liquid streams of aqueous solution A and B are each discharged independently into said elongated mixing receptacle at a linear velocity of less than 10,000 m/h with less than 5,000 m/h being preferred and less than 2500 m/h being particularly preferred.

The acid is largely removed during the washing process associated with the filtration process with any acid left being removed in the calcining process.

Variation in the reaction mixture pH within the ranges of present invention can together with reaction time or reaction temperature be used a condition to fine tune the pore size of the final ordered mesoporous silica material. The pore size increases slightly with increasing pH. The pore size increases more strongly with reaction temperature, but without substantially affecting the total pore volume. The pH at which the reaction is performed is preferably in the range of 2.2 to 7.8, particularly preferably in the range of 2.4 to 7.6, especially preferably in the range of 2.6 to 7.4.

In another embodiment the pH at which the reaction is carried out is preferably in the range of 2.8 to 7.2, particularly preferably in the range of 3 to 7.2, especially preferably in the range of 4 to 7, or 5 to 7, and more preferably in the range of 5 to 6.5, or in the range of 5.5 to 6.5.

Examples 49 to 51 exemplify the realization of pore sizes of 5 to 6 nm at room temperature. Larger pores are realized at higher temperatures as a result of swelling of the block copolymer micelles. The pore size also varies with the sodium content. Higher sodium content give rise to slightly larger pores.

According to a preferred embodiment of the process of to the present invention, the receptacle is optionally equipped with an in-line mixing device such as a static mixer.

In the process for producing an ordered mesoporous silica material with a substantially uniform pore size, according to the present invention, the mixture resulting from the liquid streams of solution A and solution B impinging may be stirred with a stirring speed in the range of 100 to 700 rpm.

It further has been demonstrated that COK-10 materials can be produced in reaction mixtures with a pH greater than 2 and less than 8 under room temperature conditions or under low temperature conditions.

The process conditions can be tuned to achieve ordered mesoporous silica materials with pore sizes selected from the range 4 to 30 nm, preferably selected from the range 7 to 30 nm, particularly preferably selected from a range 10 to 30 nm, yet more preferably selected from a range 10 to 30 nm, and especially selected from the range of 4 to 12 nm, or of 4 to 14 nm, or of 6 to 14 nm.

The aqueous solution 1 is preferably an aqueous sodium silicate solution with at least 10% by weight of sodium hydroxide and at least 27% by weight of silica.

It will be apparent to those skilled in the art that various modifications and variations can be made in the amount of reagents or of intermediate such as the amphiphilic polymers whereunder Pluronic P123, or such as the tetraalkylammonium cation, in particular the tetrapropylammonium hydroxide or in the conditions of temperature, mixing speed or reaction time of the process of present invention and in construction of the system and method without departing from the scope or spirit of the invention. Such variations can be fined tuned to manufacture the narrow pore size distribution mesoporous materials of present invention with a desired maximum pore size within the range of 7 to 30 nm.

According to a preferred embodiment of the process, according to the present invention, the residence time in the elongated mixing receptacle (conduit, tube) is in the range of 1 to 100 minutes, with a residence time of 1 to 100 s being preferred and a residence time of 1 to 10 s being particularly preferred.

According to a preferred embodiment of the process, according to the present invention, the ordered mesoporous silica material with a substantially uniform pore size is itself produced in the absence of radiation, although radiation may be used in loading said ordered mesoporous silica material with a bioactive species.

According to a preferred embodiment of the process, according to the present invention, the ordered mesoporous silica material with a substantially uniform pore size is itself produced in the absence of microwave radiation, although microwave radiation may be used in loading said ordered mesoporous silica material with a bioactive species.

According to a preferred embodiment of the process, according to the present invention the process further comprises loading of said ordered mesoporous silica material with a bioactive species.

According to a preferred embodiment of the process, according to the present invention, the process further comprises solidly dispersing a practically insoluble drug in said ordered mesoporous silica material.

According to a further preferred embodiment of the process, according to the present invention, the process is a continuous process.

### Poly(alkylene oxide) triblock copolymer

The poly(alkylene oxide) (A)ₙ(B)ₘ(A)ₙ triblock copolymer is preferably a poly(ethylene oxide)-poly(alkylene oxide)-poly(ethylene oxide) triblock copolymer, e.g. a Pluronic surfactant see Table 2 below, or a poly(alkylene oxide)-polyethylene oxide)-polyalkylene oxide triblock copolymer, e.g. a reverse Pluronic surfactant see Table 2 below, wherein the alkylene oxide moiety has at least 3 carbon atoms, for instance a propylene oxide or butylene oxide moiety, more preferably such triblock copolymers wherein the number of ethylene oxide moieties in each block is at least 5 and/or wherein the number of alkylene oxide moieties is at least 15 and if in the central block is particularly preferably at least 30.

The poly(alkylene oxide) triblock copolymer Pluronic P123 with the composition EO₂₀PO₇₀EO₂₀ (wherein EO stands for ethylene oxide, and PO stands for propylene oxide) with a formula of HO(CH₂CH₂O)₂₀(CH₂CH(CH₃)O)₇₀(CH₂CH₂O)₂₀H with a molecular weight of about 5800 and Pluronic® 17R4 with the composition (PO)₁₄(EO)₂₄(P0)₁₄ with a formula HO(CH₂CH(CH₃)O)₁₄(CH₂CH₂O)₂₄(CH₂CH(CH₃)O)₁₄H with a molecular weight of about 2700 with a terminal secondary hydroxyl group are particularly preferred.

TABLE 2

| | A | n | B | m | molecular weight |
|---|---|---|---|---|---|
| Pluronic ® 10R5 | PO* | 8 | EO** | 22 | 1950 |
| Pluronic ® 17R2 | PO* | 14 | EO** | 10 | 2150 |
| Pluronic ® 17R4 | PO* | 14 | EO** | 24 | 2650 |
| Pluronic ® 17R8 | PO* | 14 | EO** | 154 | 8500 |
| Pluronic ® 25R1 | PO* | 21 | EO** | 6 | 2800 |
| Pluronic ® 25R2 | PO* | 21 | EO** | 14 | 3100 |
| Pluronic ® 25R4 | PO* | 19 | EO** | 33 | 3600 |
| Pluronic ® 25R5 | PO* | 21 | EO** | 56 | 5000 |
| Pluronic ® 31R1 | PO* | 26 | EO** | 5 | 3250 |
| Pluronic ® 31R2 | PO* | 26 | EO** | 19 | 3875 |
| Pluronic ® 31R4 | PO* | 26 | EO** | 28 | 5150 |
| Pluronic ® L31 | EO** | 1 | PO* | 17 | 1100 |
| Pluronic ® L35 | EO** | 11 | PO* | 16 | 1900 |
| Pluronic ® F38 | EO** | 43 | PO* | 16 | 4700 |
| Pluronic ® L43 | EO** | 6 | PO* | 22 | 1850 |
| Pluronic ® L44 | EO** | 12 | PO* | 20 | 2200 |
| Pluronic ® L61 | EO** | 2 | PO* | 31 | 2000 |
| Pluronic ® L62 | EO** | 6 | PO* | 34 | 2450 |
| Pluronic ® L63 | EO** | 9 | PO* | 32 | 2650 |
| Pluronic ® L64 | EO** | 13 | PO* | 30 | 2900 |
| Pluronic ® P65 | EO** | 20 | PO* | 30 | 3400 |
| Pluronic ® F68 | EO** | 80 | PO* | 30 | 8400 |
| Pluronic ® F77 | EO** | 52 | PO* | 34 | 6600 |
| Pluronic ® L81 | EO** | 3 | PO* | 42 | 2750 |
| Pluronic ® P84 | EO** | 19 | PO* | 43 | 4200 |
| Pluronic ® P85 | EO** | 26 | PO* | 39 | 4600 |
| Pluronic ® F87 | EO** | 64 | PO* | 37 | 7700 |
| Pluronic ® F88 | EO** | 100 | PO* | 39 | 11400 |
| Pluronic ® L92 | EO** | 8 | PO* | 50 | 3650 |
| Pluronic ® F98 | EO** | 118 | PO* | 45 | 13000 |
| Pluronic ® L101 | EO** | 4 | PO* | 59 | 3800 |
| Pluronic ® P103 | EO** | 17 | PO* | 56 | 4950 |
| Pluronic ® P104 | EO** | 27 | PO* | 61 | 5900 |
| Pluronic ® P105 | EO** | 37 | PO* | 56 | 6500 |
| Pluronic ® F108 | EO** | 129 | PO* | 56 | 14600 |
| Pluronic ® L121 | EO** | 5 | PO* | 70 | 4400 |
| Pluronic ® P123 | EO** | 20 | PO* | 70 | 5800 |
| Pluronic ® F127 | EO** | 106 | PO* | 70 | 12600 |

*PO = propylene oxide
**EO = ethylene oxide

Typically a SiO₂ to Pluronic® P123 ratio is in the range of 1:30 to 1:100, or 1:50 to 1:80, or 1:60 to 1:70, e.g. said ration is 1:65.

### Acids

Acids with a pKa of less than 2 suitable for acidifying the reaction mixtures include hydrochloric acid, hydrobromic acid, sulfuric acid, nitric acid, oxalic acid, cyclamic acid, maleic acid, methanesulfonic acid, ethanesulfonic acid, benzenesulfonic acid, and p-toluenesulfonic acid. Further such acids are listed in Table 3.

TABLE 3

| acid | pKa | acid | pKa |
|---|---|---|---|
| trifluoromethane- | −13 | trifluoroacetic acid | 0.0 |
| sulfonic acid | | | |
| hydroiodic acid | <1 | trichloroacetic acid | 0.77 |
| hydrobromic acid | <1 | chromic acid | 0.74 |
| perchloric acid | −7 | iodic acid | 0.80 |
| hydrochloric acid | −4 | oxalic acid | 1.23 |
| chloric acid | <1 | dichloroacetic acid | 1.25 |
| sulfuric acid | −3 | sulfurous acid | 1.81 |
| benzenesulfonic acid | −2.5 | maleic acid | 1.83 |
| methanesulfonic acid | −2 | cyclamic acid | 1.90 |
| toluenesulfonic acid | −1.76 | chlorous acid | 1.96 |
| nitric acid | −1 | | |

Hydrochloric acid is a preferred acid for acidifying the reaction mixtures.

### Silica

The source of silica for the synthesis of ordered mesoporous material can be a monomeric source, such as the silicon alkoxides. TEOS and TMOS are typical examples of silicon alkoxides. Alternatively, alkaline silicate solutions such as waterglass can be used as silicon source. Kosuge et al. demonstrated the use of water-soluble sodium silicate for synthesizing SBA-15 type material [Kosuge et al. Chemistry of Materials, (2004), 16, 899-905]. In materials called Zeotiles, the silica is pre-assembled in zeolite-like nanoslabs that are assembled at the meso-scale into three-dimensional mosaic structures [Kremer et al. Adv. Mater. 20 (2003) 1705].

### Ordered Mesoporous Silica Materials (COK-10)

The present invention also concerns an ordered mesoporous silica material obtained by a process of synthesis at a mild pH condition between pH 2 and pH 8 (the pH in the final reaction mixture) whereby the reaction mixture is eventually free of an aromatic hydrocarbon such as 1,2,4-trimethylbenzene. Self-assembling of such materials can be obtained after the addition of a tetraalkylammonium cation, preferably tetrapropylammonium or a tetramethylammonium, as tetrapropylammonium hydroxide or tetramethylammonium hydroxide to reaction mixtures in mild pH conditions for instance a mild pH condition between pH 2 and pH 8, or a mild pH condition between pH 2.2 and pH 7.8, or a mild pH condition between pH 2.4 and pH 7.6, or a mild pH condition between pH 2.6 and pH 7.4, or a mild pH condition between pH 2.8 and pH 7.2, or a mild pH condition between pH 3 and pH 7.2, or a mild pH condition between pH 4 and pH 7, or a mild pH condition between pH 5 and pH 6.5.

The present invention also concerns an ordered mesoporous material that has a narrow mesopore size distribution around a maximum pore size selected from the range of 7 to 30 nm, 10 to 30 nm, 12 to 30 nm, 14 to 30 nm, 16 to 30 nm, 16 to 25 nm or 15 to 20 nm and which is obtained by a synthesis process under mild pH conditions i.e. a pH greater than 2 and less than 8 in the final reaction mixture, the reaction mixture being free of an aromatic hydrocarbon such as 1,2,4-trimethylbenzene. Such ordered mesoporous silica materials obtained by this process are characterized in that they have a narrow mesopore size distribution around a maximum pore size selected from the size values of 6 nm, 8 nm, 10 nm, 12 nm, 14 nm, 16 nm, 18 nm, 20 nm, 22 nm, 24 nm, 26 nm, 28 nm or 30 nm.

### Process for Self-Assembling a 2D-Hexagonal Ordered Mesoporous Silica Material with a Substantially Uniform Pore Size

Aspects of the present invention are also realized by a process for self-assembling a 2D-hexagonal ordered mesoporous silica material with a substantially uniform pore size in the range of 4 to 12 nm comprising the steps of: preparing an aqueous solution 1 comprising an alkali silicate solution; preparing an aqueous solution 3 comprising a poly(alkylene oxide) triblock copolymer and a buffer with a pH greater than 2 and less than 8, said buffer having an acid and a base component; adding said aqueous alkali silicate solution to said aqueous solution giving a pH greater than 2 and less than 8 and allowing a reaction between the components to take place at a temperature in the range of 10 to 100° C., and filtering off, drying and calcinating the reaction product to produce said 2D-hexagonal ordered mesoporous silica material with a substantially uniform pore size.

Variation in the reaction mixture pH within the ranges of present invention can together with reaction time or reaction temperature be used a condition to fine tune the pore size of the final ordered mesoporous silica material. The pore size increases slightly with increasing pH. The pH at which the reaction is performed is preferably in the range of 2.2 to 7.8, particularly preferably in the range of 2.4 to 7.6, especially preferably in the range of 2.6 to 7.4.

In another embodiment the pH at which the reaction is carried out is preferably in the range of 2.8 to 7.2, particularly preferably in the range of 3 to 7.2, especially preferably in the range of 4 to 7 and particularly especially preferably in the range of 5 to 6.5.

In the process for self-assembling a 2D-hexagonal ordered mesoporous silica material with a substantially uniform pore size, according to the present invention, the stirring speed is preferably in the range of 100 to 700 rpm.

The poly(alkylene oxide) triblock copolymer is preferably Pluronic P123.

The aqueous solution 1 is preferably an aqueous sodium silicate solution with at least 10% by weight of sodium hydroxide and at least 27% by weight of silica.

It will be apparent to those skilled in the art that various modifications and variations can be made in the amount of reagents, in the pH, temperature, mixing speed or reaction time of the process of the present invention and in construction of the system and method without departing from the scope or spirit of the invention. Such variations can be fined tuned to manufacture the narrow pore size distribution mesoporous materials of present invention with a desired maximum pore size within the range of 4 to 12 nm.

### Acids with a pKa Value in the Range of 3 to 9

Suitable acids with a pKa values in the range of ca. 3 to ca. 9 include those given in the table 4 below.

TABLE 4

| | HA | pKa |
|---|---|---|
| citric acid | H₃C₆H₅O₇ | 3.14 |
| ascorbic acid | H₂C₆H₆O₆ | 4.10 |
| succinic acid | (—CH₂COOH)₂ | 4.16 |
| benzoic acid | C₆H₅COOH | 4.19 |
| glutaric acid | HOOC(CH₂)₃—COOH | 4.31 |
| p-hydroxy- | | 4.48 |
| benzoic acid | | |
| acetic acid | CH₃COOH | 4.75 |
| citric acid | H₂C₆H₅O₇⁻ | 4.77 |
| tartaric acid | ⁻OOCCH(OH)—CH(OH)COOH | 4.8 |
| propionic acid | C₂H₅COOH | 4.87 |
| succinic acid | HOOCCH₂CH₂—COO⁻ | 5.61 |
| malonic acid | ⁻OOCCH₂COOH | 5.69 |
| carbonic acid | H₂CO₃ | 6.35 |
| citric acid | HC₆H₅O₇²⁻ | 6.39 |
| phosphoric acid | H₂PO₄²⁻ | 7.21 |
| boric acid | H₃BO₃ | 9.27 |

In a preferred embodiment of the process for self-assembling a 2D-hexagonal ordered mesoporous silica material with a substantially uniform pore size, according to the present invention, the acids has a pKa value in the range of 4 to 7. Adding aqueous solution 1 to aqueous solution 4 results in a pH greater than 2 and less than 8 being realized which is within a range of 1.5 pH units above and 1.5 pH units below a pH having the same numerical value as a pKa of the acid with a pKa in the range of 3 to 9 i.e. a buffer solution is produced due to the effect of mixing the alkali in the alkali silicate solution and acid with a pKa in the range of 3 to 9. Citric acid, acetic acid, succinic acid and phosphoric acid are particularly preferred, which upon mixing aqueous solutions 1 and 4 give a citrate/citric acid buffer, an acetate/acetic acid buffer, a succinate/succinic acid buffer or an H₂PO₄/HPO₄⁻ buffer respectively.

In a preferred embodiment of the process for self-assembling a 2D-hexagonal ordered mesoporous silica material with a substantially uniform pore size, according to the present invention, the acids has a pKa value in the range of 4 to 7,

### Buffers with a pH Greater than 2 and Less than 8

The pH greater than 2 and less than 8 is preferably in the pH zone for the acid component of the buffer i.e. within the range of 1.5 pH units above and 1.5 pH units below the pH having the same numerical value as the pKa of the acid component of the buffer, with a pH range of 1.2 pH units above and 1.2 pH units below the pH having the same numerical value as the pKa of the acid component being particularly preferred and a pH range of 1.0 pH units above and 1.0 pH units below the pH having the same numerical value as the pKa of the acid component being especially preferred.

Buffers are a mixture of weak acids and salt of the weak acids or a mixture of salts of weak acids. Preferred buffers are buffers on the basis of polyacids/salts of salts of polyacids which have multiple pKa's within the range of 2 to 8 such as citric acid/citrate salt buffers with buffer zones round each pKa which overlap to cover the whole range between 2.0 and 7.9: 3.14±1.5, 4.77±1.5 and 6.39±1.5 respectively; and succinic acid/succinic acid salt buffers with buffer zones round each pKa which overlap to cover the whole range between 2.66 and 7.1: 4.16±1.5 and 5.61±1.5 respectively.

Preferred buffers with a pH greater than 2 and less than 8 include sodium citrate/citric acid buffers with a pH range of 2.5 to 7.9, sodium acetate/acetic acid buffers with a pH range of 3.2 to 6.2, Na₂HPO₄/citric acid buffers with a pH range of 3.0 to 8.0, HCl/sodium citrate buffers with a pH range of 1 to 5 and Na₂HPO₄/NaH₂PO₄ buffers with a pH range of 6 to 9.

The sodium/citric acid buffer preferably has a sodium citrate:citric acid weight ratio in the range of 0.1:1 to 3.3:1.

### Drugs

The Biopharmaceutical Classification System (BCS) is a framework for classifying drug substances based on their aqueous solubility and intestinal permeability (Amidon, G. L., Lennernäs H., Shah V. P., and Crison J. R., “A Theoretical Basis For a Biopharmaceutics Drug Classification: The Correlation of In Vitro Drug Product Dissolution and In Vivo Bioavailability”, Pharmaceutical Research, 12: 413-420 (1995) and Adkin, D. A., Davis, S. S., Sparrow, R. A., Huckle, P. D. and Wilding, I. R., 1995. The effect of mannitol on the oral bioavailability of cimetidine. J. Pharm. Sci. 84, pp. 1405-1409).

The Biopharmaceutical Classification System (BCS), originally developed by G. Amidon, separates pharmaceuticals for oral administration into four classes depending on their aqueous solubility and their permeability through the intestinal cell layer. According to the BCS, drug substances are classified as follows:

### Class I—High Permeability, High Solubility

### Class II—High Permeability, Low Solubility

### Class III—Low Permeability, High Solubility

### Class IV—Low Permeability, Low Solubility

The interest in this classification system stems largely from its application in early drug development and then in the management of product change through its life-cycle. In the early stages of drug development, knowledge of the class of a particular drug is an important factor influencing the decision to continue or stop its development. The present delivery form and the suitable method of present invention can change this decision point by providing better bioavailability of Class 2 drugs of the BCS system.

The solubility class boundary is based on the highest dose strength of an immediate release (“IR”) formulation and a pH-solubility profile of the test drug in aqueous media with a pH range of 1 to 7.5. Solubility can be measured by the shake-flask or titration method or analysis by a validated stability-indicating assay. A drug substance is considered highly soluble when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water. The permeability class boundary is based, directly, on measurements of the rate of mass transfer across human intestinal membrane, and, indirectly, on the extent of absorption (fraction of dose absorbed, not systemic bioavailability) of a drug substance in humans. The extent of absorption in humans is measured using mass-balance pharmacokinetic studies; absolute bioavailability studies; intestinal permeability methods; in vivo intestinal perfusion studies in humans; and in vivo or in situ intestinal perfusion studies in animals. In vitro permeation experiments can be conducted using excised human or animal intestinal tissue and in vitro permeation experiments can be conducted with epithelial cell monolayers. Alternatively, nonhuman systems capable of predicting the extent of drug absorption in humans can be used (e.g., in vitro epithelial cell culture methods). In the absence of evidence suggesting instability in the gastrointestinal tract, a drug is considered highly soluble when 90% or more of an administered dose, based on a mass determination or in comparison to an intravenous reference dose, is dissolved. ‘FDA guidance states pH 7.5, ICH/EU guidance states pH 6.8. An immediate release drug product is considered rapidly dissolving when no less than 85% of the labeled amount of the drug substance dissolves within 30 minutes, using USP Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes. Based on the BCS, low-solubility compounds are compounds whose highest dose is not soluble in 250 mL or less of aqueous media from pH 1.2 to 7.5 at 37° C. See Cynthia K. Brown, et al., “Acceptable Analytical: Practices for Dissolution Testing of Poorly Soluble Compounds”, Pharmaceutical Technology (December 2004). An immediate release (IR) drug product is considered rapidly dissolving when no less than 85% of the labeled amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

A drug substance is considered highly permeable when the extent of absorption in humans is determined to be greater than 90% of an administered dose, based on mass-balance or in comparison to an intravenous reference dose. The permeability class boundary is based, directly, on measurements of the rate of mass transfer across human intestinal membrane, and, indirectly, on the extent of absorption (fraction of dose absorbed, not systemic bioavailability) of a drug substance in humans. The extent of absorption in humans is measured using mass-balance pharmacokinetic studies; absolute bioavailability studies; intestinal permeability methods; in viva intestinal perfusion studies in humans; and in vivo or in situ intestinal perfusion studies in animals. In vitro permeation experiments can be conducted using excised human or animal intestinal tissue and in vitro permeation experiments can be conducted with epithelial cell monolayers. Alternatively, nonhuman systems capable of predicting the extent of drug I absorption in humans can be used (e.g., in vitro epithelial cell culture methods). A drug substance is considered highly permeable when the extent of absorption in humans is determined to be greater than 90% of an I administered dose, based on mass-balance or in comparison to an intravenous reference dose. A drug substance is considered to have low permeability when the extent of absorption in humans is determined to be less than 90% of an administered dose, based on mass-balance or in comparison to an intravenous reference dose. An IR drug product is considered rapidly dissolving when no less than 85% of the labeled amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

BCS Class II Drugs are drugs that are particularly insoluble, or slow to dissolve, but that readily are absorbed from solution by the lining of the stomach and/or the intestine. Hence, prolonged exposure to the lining of the GI tract is required to achieve absorption. Such drugs are found in many therapeutic classes. Class II drugs are particularly insoluble or slow to dissolve, but readily are absorbed from solution by the lining of the stomach and/or the intestine. Prolonged exposure to the lining of the GI tract is required to achieve absorption. Such drugs are found in many therapeutic classes. A class of particular interest is antifungal agents, such as itraconazole. Many of the known Class II drugs are hydrophobic, and have historically been difficult to administer. Moreover, because of the hydrophobicity, there tends to be a significant variation in absorption depending on whether the patient is fed or fasted at the time of taking the drug. This in turn can affect the peak level of serum concentration, making calculation of dosage and dosing regimens more complex. Many of these drugs are also relatively inexpensive, so that simple formulation methods are required and some inefficiency in yield is acceptable.

In the preferred embodiment of present invention the drug is intraconazole or a related drug, such as fluoconazole, terconazole, ketoconazole, and saperconazole.

Itraconazole is a Class II medicine used to treat fungal infections and is effective against a broad spectrum of fungi including dermatophytes (tinea infections), candida, malassezia, and chromoblastomycosis. Itraconazole works by destroying the cell wall and critical enzymes of yeast and other fungal infectious agents. Itraconazole can also decrease testosterone levels, which makes it useful in treating prostate cancer and can reduce the production of excessive adrenal corticosteroid hormones, which makes it is useful for Cushing's syndrome. Itraconazole is available in capsule and oral I solution form. For fungal infections the recommended dosage of oral capsules is 200-400 mg once a day.

Itraconazole has been available in capsule form since 1992, in oral I solution form since 1997, and in an intravenous formulation since 1999. Since Itraconazole is a highly lipophilic compound, it achieves high concentrations in fatty tissues and purulent exudates. However, its penetration into aqueous fluids is very limited. Gastric acidity and food heavily influence the absorption of the oral formulation (Bailey, et al., Pharmacotherapy, 10: 146-153 (1990)). The absorption of itraconazole oral capsule is variable and unpredictable, despite having a bioavailability of 55%.

Other suitable drugs include Class II anti-infective drugs, such as griseofulvin and related compounds such as griseoverdin; some anti-malaria drugs (e.g. Atovaquone); immune system modulators (e.g. cyclosporine); and cardiovascular drugs (e.g. digoxin and spironolactone); and ibuprofen. In addition, sterols or steroids may be used. Drugs such as Danazol, carbamazopine, and acyclovir may also be loaded into the mesoporous materials of present invention and further be manufactured into a pharmaceutical composition.

Danazol is derived from ethisterone and is a synthetic steroid. Danazol is designated as 17a-Pregna-2,4-dien-20-yno[2,3-d]-isoxazol-17-ol, has the formula of C₂₂H₂₇NO₂, and a molecular weight of 337.46. Danazol is a synthetic steroid hormone resembling a group of natural hormones (androgens) that are found in the body. Danazol is used in the treatment of endometriosis. It is also useful in the treatment of fibrocystic breast disease and hereditary angioedema. Danazol works to reduce estrogen levels by inhibiting the production of hormones called gonadotrophins by the pituitary gland. Gonadotrophins normally stimulate the production of sex hormones such as estrogen and progestogen, which are responsible for body processes such as menstruation and ovulation. Danazol is administered orally, has a bioavailability that is not directly dose-related, and a half-life of 4-5 hours. Dosage increases in danazol are not proportional to increases in plasma concentrations. It has been shown that doubling the dose may yield only a 30-40% increase in I plasma concentration. Danazol peak concentrations occur within 2 hours, but the therapeutic effect usually does not occur for approximately 6-8 weeks I after taking daily doses.

Acyclovir is a synthetic nucleoside analogue that acts as an antiviral agent. Acyclovir is available for oral administration in capsule, tablet and suspension forms. It is a white, crystalline powder designated as 2-amino-1,9-dihydro-9-[(2-hydroxyethoxy)methyl]-6H-purin-6-one, has an empirical formula of C₈H₁₁N₅O₃ and a molecular weight of 225. Acyclovir may also be loaded into the mesoporous materials of present invention and further be manufactured into a pharmaceutical composition

Acyclovir has an absolute bioavailability of 20% at a 200 mg dose given every 4 hours, with a half-life of 2.5 to 3.3 hours. In addition, the bioavailability decreases with increasing doses. Despite its low bioavailability, acyclovir is highly specific in its inhibitory activity of viruses due to its high affinity for thymidine kinase (TK) (encoded by the virus). TK converts acyclovir into a nucleotide analogue, which prevents replication of viral DNA by inhibition and/or inactivation of the viral DNA polymerase, and through termination of the growing viral DNA chain.

Carbamazepine is used in the treatment of psychomotor epilepsy, and as an adjunct in the treatment of partial epilepsies. It can also relieve or diminish pain that is associated with trigeminal neuralgia. Carbamazepine given as a monotherapy or in combination with lithium or neuroleptics has also been found useful in the treatment of acute mania and the prophylactic treatment of bipolar disorders. Carbamazepine may also be loaded into the mesoporous materials of present invention and further be manufactured into a pharmaceutical composition

Carbamazepine is a white to off-white powder, is designated as 5H dibenz[b,flazepine-5-carboxamide, and has a molecular weight of 236.77. It is practically insoluble in water and soluble in alcohol and acetone. The absorption of Carbamazepine is relatively slow, despite a bioavailability of 89% for the tablet form. When taken in a single oral dose, the Carbamazepine tablets and chewable tablets yield peak plasma concentrations of unchanged Carbamazepine within 4 to 24 hours. The therapeutic range for the steady-state plasma concentration of Carbamazepine generally lies between 4 and 10 mcg/mL.

Other representative Class II compounds are antibiotics to kill Helicobacter pylori include amoxicillin, tetracyline and metronidazole or therapeutic agents including acid suppressants (H2 blockers include cimetidine, ranitidine, famotidine, and nizatidine; Proton pump inhibitors include omeprazole, lansoprazole, rabeprazole, esomeprazole, and pantoprozole), mucosal defense enhancing agent (bismuth salts; bismuth subsalicylate) and/or mucolytic agents (megaldrate). These above mentioned species may also be loaded into the mesoporous materials of present invention and further be manufactured into a pharmaceutical composition.

Many of the known Class II drugs are hydrophobic, and have historically been difficult to administer. Moreover, because of the hydrophobicity, there tends to be a significant variation in absorption depending on whether the patient is fed or fasted at the time of taking the drug. This in turn can affect the peak level of serum concentration, making calculation of dosage and dosing regimens more complex. Many of these drugs are also relatively inexpensive, so that simple formulation methods are required and some inefficiency in yield is acceptable.

In a preferred embodiment of present invention, the drug is intraconazole and its relatives fluoconazole, terconazole, ketoconazole, and saperconazole of which such species can is loaded into the mesoporous materials of present invention and further be manufactured into a pharmaceutical composition

Itraconazole is a Class II medicine used to treat fungal infections and is effective against a broad spectrum of fungi including dermatophytes (tinea infections), candida, malassezia, and chromoblastomycosis. Itraconazole works by destroying the cell wall and critical enzymes of yeast and other fungal infectious agents. Itraconazole can also decrease testosterone levels, which makes it useful in treating prostate cancer and can reduce the production of excessive adrenal corticosteroid hormones, which makes it useful for Cushing's syndrome. Itraconazole is available in capsule and oral solution form. For fungal infections the recommended dosage of oral capsules is 200-400 mg once a day. Itraconazole has been available in capsule form since 1992, in oral solution form since 1997, and in an intravenous formulation since 1999. Since itraconazole is a highly lipophilic compound, it achieves high concentrations in fatty tissues and purulent exudates. However, its penetration into aqueous fluids is very limited. Gastric acidity and food heavily influence the absorption of the oral formulation (Bailey, et al., Pharmacotherapy, 10: 146-153 (1990)). The absorption of itraconazole oral capsule is variable and unpredictable, despite having a bioavailability of 55%.

Other Class II drugs include anti-infective drugs such as sulfasalazine, griseofulvin and related compounds such as griseoverdin; some anti malaria drugs (e.g. Atovaquone); immune system modulators (e.g. cyclosporine); and cardiovascular drugs (e.g. digoxin and spironolactone); and ibuprofen (analgesic); ritonavir, nevirapine, lopinavir (antiviral); clofazinine (leprostatic); diloxanide furoate (anti-amebic); glibenclamide (anti-diabetes); nifedipine (anti-anginal); spironolactone (diuretic); steroidal drugs such as Danazol; carbamazepine, and anti-virals such as acyclovir. These species can loaded into the mesoporous materials of present invention and further be manufactured into a pharmaceutical composition

Danazol is derived from ethisterone and is a synthetic steroid. Danazol is designated as 17a-Pregna-2,4-dien-20-yno[2,3-d]-isoxazol-17-ol, has the formula of C₂₂H₂₇NO₂, and a molecular weight of 337.46. Danazol is used in the treatment of endometriosis, fibrocystic breast disease and hereditary angioedema. Danazol is administered orally, has a bioavailability that is not directly dose-related, and a half-life of 4-5 hours. Dosage increases in danazol are not proportional to increases in plasma concentrations. It has been shown that doubling the dose may yield only a 30-40% increase in plasma concentration. Danazol peak concentrations occur within 2 hours, but the therapeutic effect usually does not occur for approximately 6-8 weeks after taking daily doses.

Acyclovir is a synthetic nucleoside analogue that acts as an antiviral agent. Acyclovir is available for oral administration in capsule, tablet, and suspension forms. It is a white, crystalline powder designated as 2-amino-1,9-dihydro-9-[(2-hydroxyethoxy)methyl]-6H-purin-6-one, has an empirical formula of C₈H₁₁N₅O₃ and a molecular weight of 225. Acyclovir has an absolute bioavailability of 20% at a 200 mg dose given every 4 hours, with a half-life of 2.5 to 3.3 hours. The bioavailability decreases with increasing doses. Despite its low bioavailability, acyclovir is highly specific in its inhibitory activity of viruses due to its high affinity for thymidine kinase (TK) (encoded by the virus). TK converts acyclovir into a nucleotide analogue which prevents replication of viral DNA by inhibition and/or inactivation of the viral DNA polymerase, and through termination of the growing viral DNA chain. Acyclovir can be m loaded into the mesoporous materials of present invention and further be manufactured into a pharmaceutical composition

Carbamazepine is used in the treatment of psychomotor epilepsy, and as an adjunct in the treatment of partial epilepsies. It can also relieve or diminish pain that is associated with trigeminal neuralgia. Carbamazepine given as a monotherapy or in combination with lithium or neuroleptics has also been found useful in the treatment of acute mania and the prophylactic treatment of bipolar disorders. Carbamazepine is a white to off-white powder, is designated as 5H-dibenz[b,f]azepine-5-carboxamide, and has a molecular weight of 236.77. It is practically insoluble in water and soluble in alcohol and acetone. The absorption of carbamazepine is relatively slow, despite a bioavailability of 89% for the tablet form. When taken in a single oral dose, the carbamazepine tablets and chewable tablets yield peak plasma concentrations of unchanged carbamazepine within 4 to 24 hours. The therapeutic range for the steady-state plasma concentration of carbamazepine generally lies between 4 and 10 mcg/mL. Carbamazepine may also be loaded into the mesoporous materials of present invention and further be manufactured into a pharmaceutical composition

BCS Class IV Drugs (Low Permeability, Low Solubility) are drugs that are particularly insoluble, or slow to dissolve, in water and with poor with poor GI permeability.

Most class IV drugs are lipophilic drugs which results in their consequent poor GI permeability. Examples include acetazolamide, furosemide, tobramycin, cefuroxmine, allopurinol, dapsone, doxycycline, paracetamol, nalidixic acid, clorothiazide, tobramycin, cyclosporin, tacrolimus, and paclitaxel. Tacrolimus is a macrolide immuno-suppressant produced by Streptomyces tsukubaensis. Tacrolimus prolongs the survival of the host and transplanted graft in animal transplant models of liver, kidney, heart, bone marrow, small bowel and pancreas, lung and trachea, skin, cornea, and limb. Tacrolimus acts as an immuno-suppressant through inhibition of T-lymphocyte activation through a mechanism that is unknown. Tacrolimus has an empirical formula of C₄₄H₆₉NO 12.H₂O and a formula weight of 822.05. Tacrolimus appears as white crystals or crystalline powder. It is practically insoluble in water, freely soluble in ethanol, and very soluble in methanol and chloroform. Tacrolimus is available for oral administration as capsules or as a sterile solution for injection. Absorption of tacrolimus from the gastro-intestinal tract after oral administration is incomplete and variable. The absolute bioavailability of tacrolimus is approximately 17% at a 5 mg dose taken twice a day. Paclitaxel is a chemotherapeutic agent that displays cytotoxic and antitumor activity. Paclitaxel is a natural product obtained via a semi-synthetic process from Taxus baccata. While having an unambiguous reputation of tremendous therapeutic potential, paclitaxel has some patient-related drawbacks as a therapeutic agent. These partly stem from its extremely low solubility in water, which makes it difficult to provide in suitable dosage form. Because of paclitaxel's poor aqueous solubility, the current approved (U.S. FDA) clinical formulation consists of a 6 mg/ml solution of paclitaxel in 50% polyoxyethylated castor oil (CREMOPHOR EL®) and 50% dehydrated alcohol. Am. J. Hosp. Pharm., 48:1520-24 (1991). In some instances, severe reactions, including hypersensitivity, occur in conjunction with the CREMOPHOR® administered in conjunction with paclitaxel to compensate for its low water solubility. As a result of the incidence of hypersensitivity reactions to the commercial paclitaxel formulations and the potential for paclitaxel precipitation in the blood, the formulation must be infused over several hours. In addition, patients must be pretreated with steroids and antihistamines prior to the infusion. Paclitaxel is a white to off-white crystalline powder available in a nonaqueous solution for injection. Paclitaxel is highly lipophilic and insoluble in water. Such lipophilic drugs may also be loaded into the mesoporous materials of present invention and further be manufactured into a pharmaceutical composition.

Examples of compounds that are poorly soluble in water are poorly soluble drugs can be taken from the groups of the prostaglandines, e.g. prostaglandine E2, prostaglandine F2 and prostaglandine E1, proteinase inhibitors, e.g. indinavire, nelfinavire, ritonavire, saquinavir, cytotoxics, e.g. paclitaxel, doxorubicine, daunorubicine, epirubicine, idarubicine, zorubicine, mitoxantrone, amsacrine, vinblastine, vincristine, vindesine, dactiomycine, bleomycine, metallocenes, e.g. titanium metallocene dichloride, and lipid-drug conjugates, e.g. diminazene stearate and diminazene oleate, and generally poorly insoluble anti-infectives such as griseofulvine, ketoconazole, fluconazole, itraconazole, clindamycine, especially antiparasitic drugs, e.g chloroquine, mefloquine, primaquine, vancomycin, vecuronium, pentamidine, metronidazole, nimorazole, timidazole, atovaquone, buparvaquone, nifurtimoxe and anti-inflammatory drugs, e.g. cyclosporine, methotrexate, azathioprine. These bioactive compounds may also be loaded into the mesoporous materials of present invention and further be manufactured into a pharmaceutical composition.

### Pharmaceutical Composition

The ordered mesoporous silica materials of the present invention hosting a bioactive species such as a poorly water soluble drug or a drug that is practically insoluble in water, or an antibody fragment or a nucleotide fragment can be formulated as pharmaceutical compositions and administered to a mammalian host, such as a human patient or a domestic animal in a variety of forms adapted to the chosen route of administration, i.e., the oral, peroral, topical, orally, parenteral, rectal or other delivery routes.

The ordered mesoporous silica materials of the present invention may also host small oligonucleic acid or peptide molecules for instance such that bind a specific target molecule such as aptamers. (DNA aptamers, RNA aptamers or peptide aptamers). The mesoporous materials of present invention hosting the small oligonucleic acid or intended to host such can be used for hybridisation of such oligonucleic acids.

The ordered mesoporous materials of the present invention are particularly suitable to host and cause immediate release in watery environments of a poorly water soluble drug, a BCS Class II drug, a BCS Class IV drug or a compound that is practically insoluble in water. For example Itraconazole can be loaded into the ordered mesoporous silica materials of the present invention.

The pharmaceutical composition (preparation) according to the present invention may be produced by a method that is optionally selected from, for example, “Guide Book of Japanese Pharmacopoeia”, Ed. of Editorial Committee of Japanese Pharmacopoeia, Version No. 13, published Jul. 10, 1996 by Hirokawa publishing company The new mesoporous materials of present invention can be used to host small antibody fragments. Examples of small antibody fragments are Fv″ fragment, single-chain Fv (scFv) antibody, antibody Fab fragments, antibody Fab′ fragments, antibody fragment of heavy or light chain CDRs, or anobodies.

Washed, dried and calcined COK-10 materials loaded with a poorly water soluble bioactive species into its pores exhibit an improved releasing speed of these poorly water soluble bioactive species into a watery medium.

In view of the mild conditions under which the process of the present invention can be carried out, the process can be used to encapsulate drugs present in solution B during the synthesis process, according to the present invention.

### Loading of Ordered Mesoporous Silica Materials

A solution in solvent: 50/50 V/V dichloromethane/ethanol can be prepared for bioactive species such as 1) Itraconazole, 2) an Itraconazole derivative, 3) a triazole compound wherein the polar surface area (PSA) is in the range from 60 Å² to 200 Å², preferably from 70 Å² to 160 Å², more preferably form 80 Å² to 140 Å², yet more preferably from 90 Å² to 120 Å² and most preferably from 95 Å² to 110 Å², 4) a triazole compound with a partition coefficient (XlogP) in the range from 4 to 9, more preferably in the range from 5 to 8 and most preferably in the range from 6 to 7, 5) a triazole compound with more than 10 freely rotating bonds, 6) triazole compound with polar surface area (PSA) in the range from 80 and 200, a partition coefficient in the range from 3 and 8 and with 8 to 16 freely rotating bonds or 7) A triazole compound with a Polar Surface Area lager than 80 Å. Sonicated can be used to speed up Itraconazole dissolution process. Such solutions which can easily have an amount of 50 mg dissolved bioactive species per ml of solvent mixture is suitable for impregnation of the mesoporous materials of present invention to have the bioactive species been loaded into the pores and molecularly dispersed in the said mesoporous material.

Another solvent that is generally suitable for dissolution compounds that are practically insoluble in water or for poorly water soluble compounds is dichloromethane (CH₂Cl₂). A solution holding 50 mg of bioactive species solved in 1 ml can be used for impregnation of the mesoporous materials of present invention to load the bioactive species into the pores. But dichloromethane can be replaced by another organic (carbon-containing) solvent such as the reaction inert solvents 1,4-dioxane, tetrahydrofuran, 2-propanol, N-methyl-pyrrolidinon, chloroform, hexafluoroisopropanol and the like. Particularly suitable for replacing are the polar aprotic solvents selected of the group 1,4-Dioxane (/—CH₂—CH₂—O—CH₂—CH₂—O—\), tetrahydrofuran (/—CH₂—CH₂—O—CH₂—CH₂—\), acetone (CH₃—C(═O)—CH₃), acetonitrile (CH₃—C≡N), dimethylformamide (H—C(═O)N(CH₃)₂) or dimethyl sulfoxide (CH₃—S(═O)—CH₃) or members selected of the group of the non-polar solvents such as hexane (CH₃—CH₂—CH₂—CH₂—CH₂—CH₃), benzene (C₆H₆), toluene (C₆H₅—CH₃), diethyl ether (CH₃CH₂—O—CH₂—CH₃), chloroform (CHCl₃), ethyl acetate (CH₃—C(═O)—O—CH₂—CH₃). Moreover appropriate organic (carbon-containing) solvent for the meaning of this invention is a solvent in which the poorly water soluble bioactive species or drug is soluble or which is an organic solvent in which a poorly water soluble drug has high solubility. For instance an organic compound such as a fluorinated alcohol for instance hexafluoroisopropanol, (HFIP —(CF₃)₂CHOH) exhibits strong hydrogen bonding properties can be used to dissolve substances that serve as hydrogen-bond acceptors, such as amides and ethers, which are poorly water soluble. Bioactive species or drug compounds of the amides class contain carbonyl (C═O) and ether (N—C) dipoles arising from covalent bonding between electronegative oxygen and nitrogen atoms and electro-neutral carbon atoms, whereas the primary and secondary amides also contain two- and one N—H dipoles, respectively. The presence of a C═O dipole and, to a lesser extent a N—C dipole, allows amides to act as H-bond acceptors, which makes that HFIP is an appropriate solvent. For instance another group of organic solvent is the non-polar solvents for instance halogenated hydrocarbons (e.g. dichloromethane, chloroform, chloroethane, trichloroethane, carbon tetrachloride, etc.), where under the most preferred is dichloromethane (DCM) or methylene chloride, which is an appropriate solvent for bioactive species or drugs such as diazepam, alpha-methyl-p-tyrosine, phencyclidine, quinolinic acid, simvastatin, lovastatin; paclitaxel, alkaloids, cannabinoids. Files and databases are available for common solvents and drug compounds (such as COSMOfiles (Trademark) of Cosmologic Gmbh & Co, GK) to the skilled man to select an appropriate solvent to load the known poorly soluble biologically active species into the ordered mesoporous oxides. For new structures drug solubility in any solvent can be calculated using thermodynamic criteria which contain basic physical properties and phase equilibrium relationships for instance by computational chemistry and fluid dynamics expert systems (T. Bieker, K. H. Simmrock, Comput. Chem. Eng. 18 (Suppl. 1) (1993) S25-S29; K. G. Joback, G. Stephanopoulos, Adv. Chem. Eng. 21 (1995) 257-311; L. Constantinou, K. Bagherpour, R. Gani, J. A. Klein, D. T. Wu, Comput. Chem. Eng. 20 (1996) 685-702; J. Gmehling, C. Moellmann, Ind. Eng. Chem. Res. 37 (1998) 3112-3123; M. Hostrup, P. M. Harper, R. Gani, Comput. Chem. Eng. 23 (1999) 1395-1414 and R. Zhao, H. Cabezas, S. R. Nishtala, Green Chemical Syntheses and Processes, ACS Symposium Series 767, American Chemical Society, Washington, D.C., 2000, pp. 230-243.) such as COSMOfrag/COSMOtherm (Trademark) of Cosmologic Gmbh & Co, GK, which interact with databases of multiple characterized molecules. Another opportunity is the availability to the skilled person of the automated drug solubility testers such as the Biomek® FX of Millipore to test without undue burden the water solubility of selected compound.

### EXAMPLES

The following examples teach the synthesis of COK-10 and COK-12 and illustrate synthesis conditions for obtaining a narrow mesopore size distribution.

### Example 1

### Synthesis of COK-10 Using TPAOH (SiO₂/TPAOH=25/1) with the pH of the Reaction Mixture Equal to 5.8

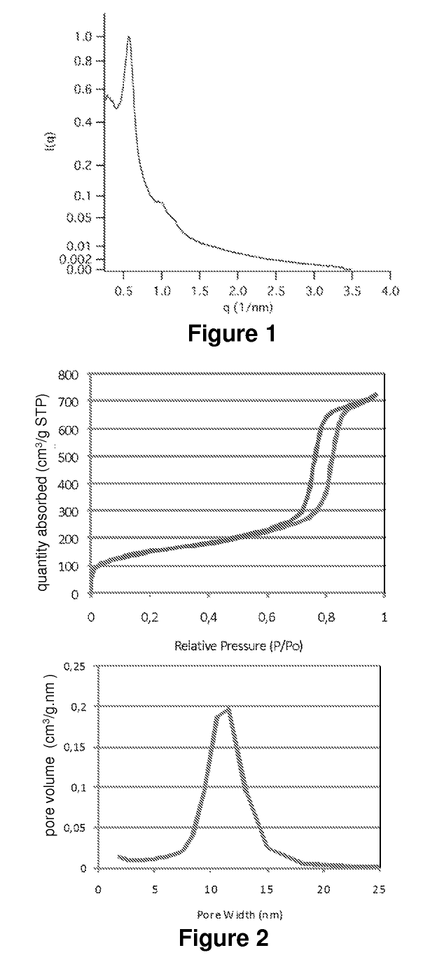

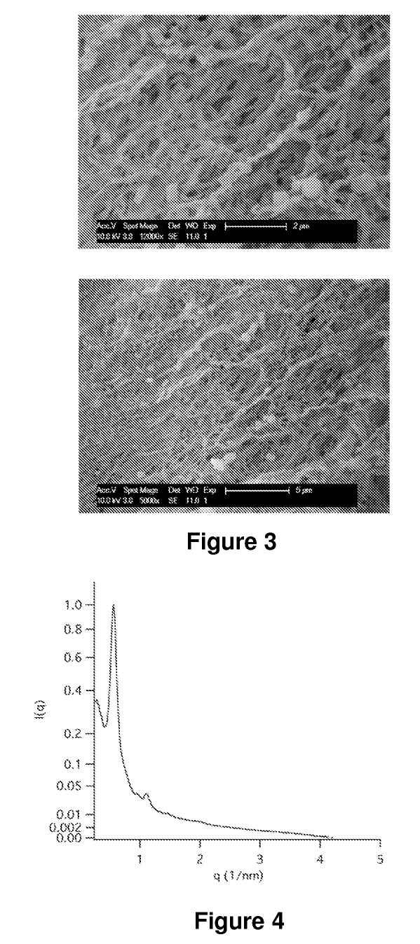

An amount of 4.181 g of Pluronic P123 surfactant (BASF) was mixed with 107.554 g of water, 12.64 g HCl solution (2.4M) and 1.8 ml of a 1M tetrapropylammonium hydroxide (TPAOH) solution (from Alpha) in a PP vessel (500 ml). This vessel was placed in an oil bath at 35° C. and stirred using a magnetic stirrer (400 rpm) overnight. In a second PP recipient 10.411 g of a sodium silicate solution (Riedel de Haën, purum, at least 10 wt. % NaOH and at least 27 wt.-% of SiO₂) was mixed with 30.029 g of water. This mixture was stirred using a magnetic stirrer (400 rpm) at room temperature for 5 minutes. The latter solution was added to the PP vessel in the oil bath. The resulting solution is stirred (400 rpm) for 5 minutes at 35° C. During this step the pH was measured to be 5.8, using a Mettler Toledo, InLab®Expert Pro pH electrode. The resulting reaction mixture was placed in a preheated oven at 35° C. for 24 h without stirring. After 24 h the temperature of the oven was raised to 90° C. and held isothermal for 24 h. The resulting reaction mixture was cooled to room temperature and vacuum filtered (particle retention 20-25 μm). The powder on the filter was washed using 300 ml of water. The resulting powder was dried in a glass recipient for 24 h at 60° C. The X-ray scattering pattern of the as-synthesized material is shown in FIG. 1. The presence of diffraction peaks reveals that the material is ordered at the meso-scale. The as-synthesized powder was transferred to porcelain plates and calcined in an air oven at 550° C. for 8 h using a heating rate of 1° C./min. The nitrogen adsorption isotherm of the calcined COK-10 material is shown in FIG. 2. The measurement was performed on a Micromeritics Tristar 3000 apparatus. Prior to measurement, the sample was pretreated at 300° C. for 10 h (ramp: 5° C./min). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 2B). The pore size is ca. 11 nm. The results from nitrogen adsorption (FIG. 2) together with X-ray scattering (FIG. 1) show that this COK-10 sample is an ordered mesoporous material. The morphology of the sample was investigated with SEM (FIG. 3). The material consists of a network of intergrown particles.

### Example 2

### Synthesis of COK-10 using TPAOH (SiO₂/TPAOH=25/1) with the pH of the Reaction Mixture Equal to 2.4

An amount of 4.162 g of Pluronic P123 surfactant was mixed with 107.093 g of water, 13.039 g HCl solution (2.4M) and 1.8 ml of a 1M TPAOH solution (from the company Alpha) in a PP vessel (500 ml). This vessel was placed in an oil bath at 35° C. and stirred using a magnetic stirrer (400 rpm) overnight. In a second PP recipient 10.441 g of a sodium silicate solution (Riedel de Haën, purum, at least 10 wt.-% NaOH and at least 27 wt.-% of SiO₂) was mixed with 30.027 g of water. This mixture was stirred using a magnetic stirrer (400 rpm) at room temperature for 5 minutes. The latter solution was added to the PP vessel in the oil bath. The resulting solution is stirred (400 rpm) for 5 minutes at 35° C. During this step the pH was measured to be 2.4, using a Mettler Toledo, InLab®Expert Pro pH electrode. The resulting reaction mixture was placed in a preheated oven at 35° C. for 24 h without stirring. After 24 h the temperature of the oven was raised to 90° C. and held isothermal for 24 h. The resulting reaction mixture was cooled to room temperature and vacuum filtered (particle retention 20-25 μm). The powder on the filter was washed using 300 ml of water. The resulting powder was dried in a glass recipient for 24 h at 60° C. Finally the powder was transferred to porcelain plates and calcined in an air oven at 550° C. for 8 h using a heating rate of 1° C./min.

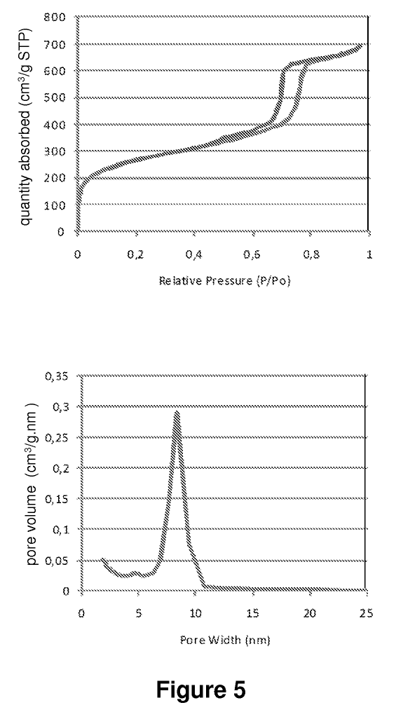

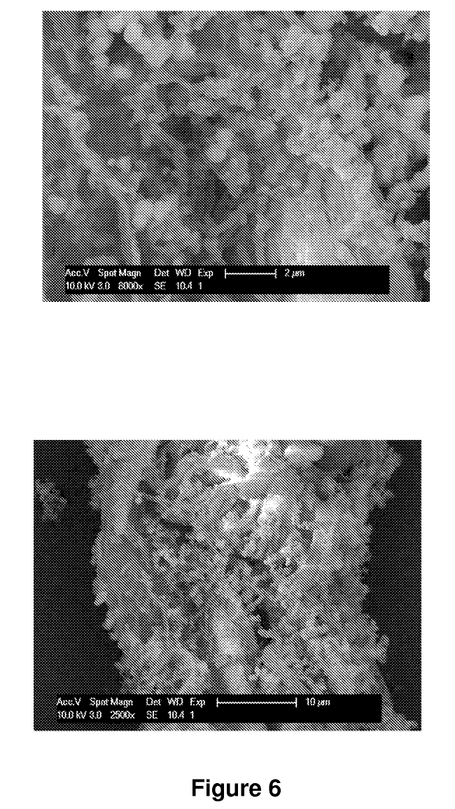

The presence of diffraction peaks at low q values in the X-ray scattering pattern of this particular COK-10 material (FIG. 4) reveals that the material is ordered at the meso-scale. The nitrogen adsorption isotherm on this sample was determined using a Micromeritics Tristar apparatus. Prior to measurement, the sample was pretreated at 300° C. for 10 h (ramp: 5° C./min). The nitrogen adsorption isotherm (FIG. 5) reveals the presence of a type IV adsorption isotherm with a hysteresis loop. The branches of the hysteresis loop are steep, which is indicative of a narrow mesopore size distribution. The mesopore size was estimated using the BJH method (FIG. 5). The pore size is around 9 nm. The morphology of the samples was investigated with SEM (FIG. 6).

### Example 3

### Synthesis of Mesoporous Material with the pH of the Reaction Mixture Equal to 6.4 Without TPAOH (Comparative Example)

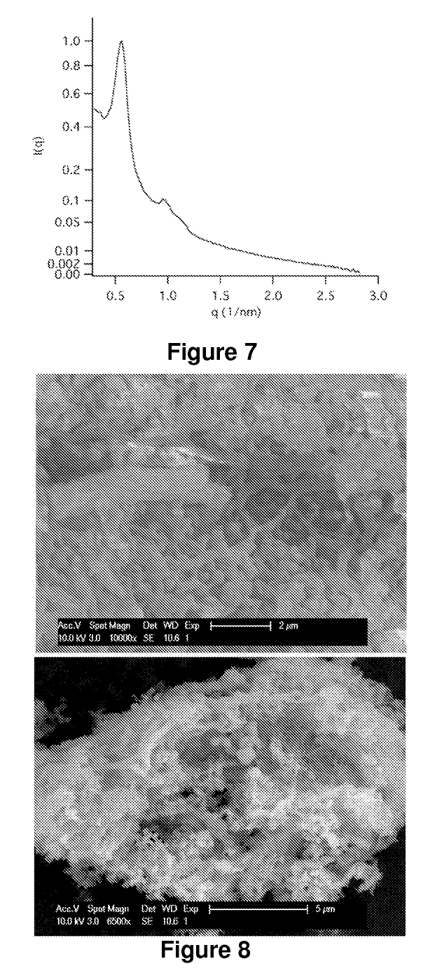

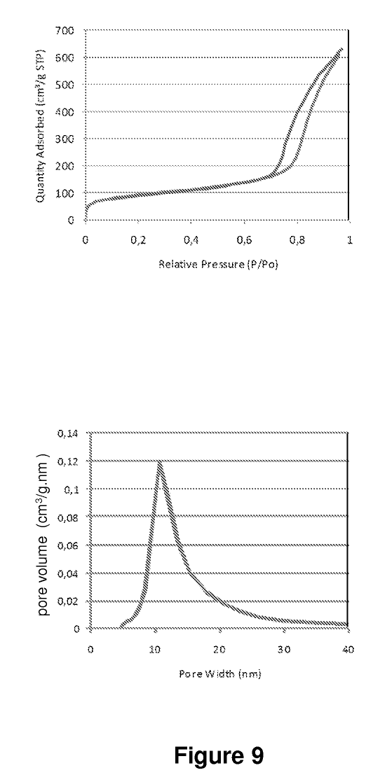

An amount of 4.212 g of Pluronic P123 surfactant was mixed with 107.592 g of water, 12.630 g HCl solution (2.4M) and 0.066 g of NaOH in a PP vessel (500 ml). This vessel was placed in an oil bath at 35° C. and stirred using a magnetic stirrer (400 rpm) overnight. In a second PP recipient 10.413 g of a sodium silicate solution (Riedel de Haën, purum, at least 10 wt.-% NaOH and at least 27 wt.-% of SiO₂) was mixed with 30.020 g of water. This mixture was stirred using a magnetic stirrer (400 rpm) at room temperature for 5 minutes. The latter solution was added to the PP vessel in the oil bath. The resulting solution was stirred (400 rpm) for 5 minutes at 35° C. During this step the pH was measured to be 6.4, using a Mettler Toledo, InLab®Expert Pro pH electrode. The resulting reaction mixture was placed in a preheated oven at 35° C. for 24 h without stirring. After 24 h the temperature of the oven was raised to 90° C. and held isothermal for 24 h. The resulting reaction mixture was cooled to room temperature and vacuum filtered (particle retention 20-25 μm). The powder on the filter was washed using 300 ml of water. The resulting powder was dried in a glass recipient for 24 h at 60° C. Finally the powder was transferred to porcelain plates and calcined in an air oven at 550° C. for 8 h using a heating rate of 1° C./min. The X-ray scattering pattern in the low angle region (FIG. 7) shows several diffraction peaks. This indicates that the material is ordered at the meso-scale. SEM pictures of this COK-10 material shown in FIG. 8 reveal the presence of aggregated particles. The nitrogen adsorption isotherm on this sample was determined using a Micromeritics Tristar apparatus (FIG. 9). Prior to the measurement, the sample was pretreated at 300° C. for 10 h (ramp: 5° C./min). The material exhibits a nitrogen adsorption isotherm with hysteresis, indicative of the presence of mesopores. The branches of the hysteresis loop do not run in parallel. The analysis of the mesopore size distribution reveals that in this sample there is a very wide variety of mesopore diameters in the range from ca. 5 to 40 nm, with a maximum at 11 nm. This example teaches that in the absence of an organic cation such as tetrapropylammonium, the ordering at the meso-scale is difficult to achieve.

### Example 4

### Synthesis of SBA-15 (Comparative Example)

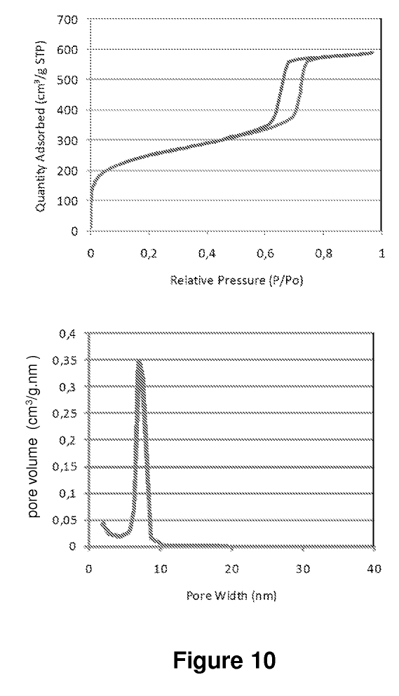

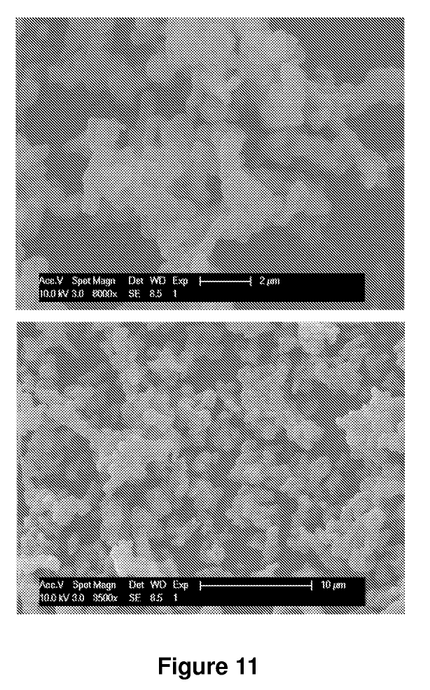

In this example a strongly acidic synthesis mixture is used. The strong acidity is obtained by using a large amount of 2M HCl solution. An amount of 4.1 g of Pluronic P123 surfactant (BASF) was mixed with 120.1 g HCl solution (2M) in a PP vessel (500 ml). This vessel was placed in an oil bath at 35° C. and stirred using a magnetic stirrer (400 rpm) overnight. In a second PP recipient 10.4 g of a sodium silicate solution (Riedel de Haën, purum, at least 10 wt.-% NaOH and at least 27 wt.-% of SiO₂) was mixed with 30.0 g of water. This mixture was stirred using a magnetic stirrer (400 rpm) at room temperature for 5 minutes. The latter solution was added to the PP vessel in the oil bath. The resulting solution is stirred (400 rpm) for 5 minutes at 35° C. The resulting reaction mixture was placed in a preheated oven at 35° C. for 24 h without stirring. After 24 h the temperature of the oven was raised to 90° C. and held isothermal for 24 h. The resulting reaction mixture was cooled to room temperature and vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min. The nitrogen adsorption isotherm of this SBA-15 is shown in FIG. 10. The obtained SBA-15 material has a pore size of ca. 8 nm. Measurement was performed on a Micromeritics Tristar 3000 apparatus. Prior to measurement, the sample was pretreated at 300° C. for 10 h (ramp: 5° C./min). A SEM picture of the obtained SBA-15 material is shown in FIG. 11. The material appears as aggregated micron size particles.

### Example 5

### Synthesis Experiment Using TPAOH (SiO₂/TPAOH=25/1) with the pH of the Reaction Mixture Equal to 11.12 (Comparative Example)

An amount of 4.043 g of Pluronic P123 surfactant was mixed with 140.335 g of water, 2.6 g HCl solution (2M) and 1.8 ml of a 1M TPAOH solution in a PP vessel (500 ml). The mixture was stirred with a magnetic stirrer (400 rpm) at room temperature. In a second PP recipient 10.428 g of a sodium silicate solution was mixed with 5.510 g of water. This mixture was stirred using a magnetic stirrer (400 rpm) at room temperature for 5 minutes. The latter solution was added to the surfactant mixture. The resulting reaction mixture was stirred (400 rpm) for 5 minutes at room temperature. During this step the pH was measured to be 11.12, using a Mettler Toledo, InLab®Expert Pro pH electrode. The reaction mixture remained a transparent gel. There was no formation of silica particles. The pH of 11.12 is outside the preferred range for synthesizing a COK-10 material.

### Example 6

### Synthesis Experiment Using TPAOH (SiO₂/TPAOH=25/1) with the pH of the Reaction Mixture Equal to 8.9 (Comparative Example)

An amount of 0.811 g of Pluronic P123 surfactant was mixed with 22.1 g of water, 2.01 g HCl solution (2.4M) and 1.8 ml of a 1M TPAOH solution in a PP vessel (60 ml). The mixture was stirred with a magnetic stirrer (400 rpm) at room temperature. In a second PP recipient 2.090 g of a sodium silicate solution was mixed with 6.261 g of water. This mixture was stirred using a magnetic stirrer (400 rpm) at room temperature for 5 minutes. The latter solution was added to the surfactant mixture. The resulting reaction mixture was stirred (400 rpm) for 5 minutes at room temperature. During this step the pH was measured to be 8.9, using a Mettler Toledo, InLab®Expert Pro pH electrode. In this synthesis the mixture remained a transparent gel. There was no formation of silica particles. This example teaches that a pH of 8.9 is outside the preferred range for COK-10 synthesis.

### Example 7

### Synthesis of COK-10 Using TPAOH (SiO₂/TPAOH=25/1) with the pH of the Reaction Mixture Equal to 5.8

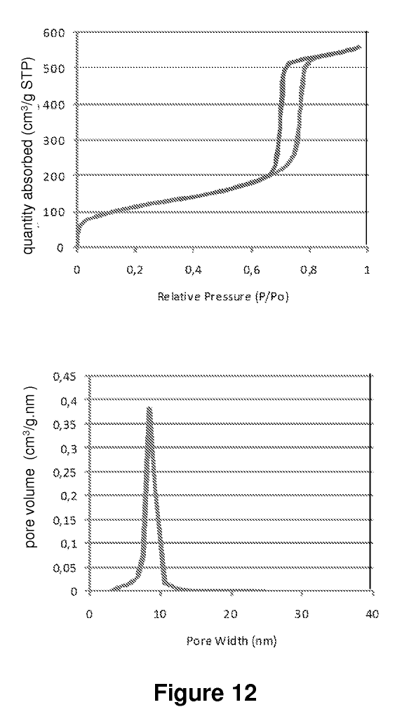

An amount of 4.140 g of Pluronic P123 surfactant was mixed with 107.55 g of water, 12.779 g HCl solution (2.4M) and 1.8 ml of a 1M TPAOH solution in a PP vessel (500 ml). The mixture was stirred with a magnetic stirrer (400 rpm) at room temperature. In a second PP recipient 10.448 g of a sodium silicate solution was mixed with 30.324 g of water. This mixture was stirred using a magnetic stirrer (400 rpm) at room temperature. The latter solution was added to the surfactant mixture. The resulting reaction mixture was stirred with a direct drive electric mixer (400 rpm) for 5 minutes. At the end of this step the pH was measured to be 5.8 using a Mettler Toledo, InLab®Expert Pro pH electrode. The resulting reaction mixture was placed in a preheated oven at 35° C. for 24 h without stirring. After 24 h the temperature of the oven was raised to 90° C. and held isothermal for 24 h. The resulting reaction mixture was cooled to room temperature and vacuum filtered (particle retention 20-25 μm). The powder on the filter was washed using 100 ml of water. The resulting powder was dried in a glass recipient for 24 h at 60° C. Finally the powder was transferred to porcelain plates and calcined in an air oven at 550° C. for 8 h using a heating rate of 1° C./min. The determination of the nitrogen adsorption isotherm was performed on a Micromeritics Tristar apparatus. Prior to measurement, the sample was pretreated at 300° C. for 10 h (ramp: 5° C./min). The nitrogen adsorption isotherm (FIG. 12) shows a hysteresis loop with parallel and steep branches typical of ordered mesoporous material. This COK-10 material has a narrow mesopore size distribution with a maximum around 9 nm (FIG. 12).

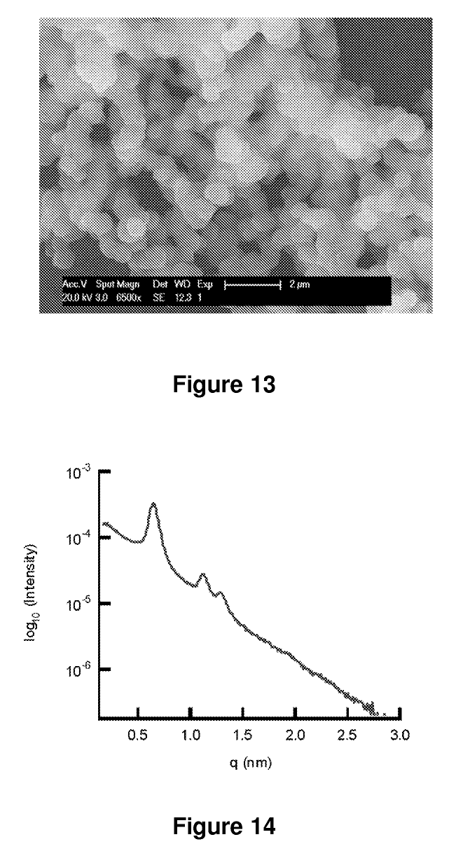

This COK-10 material consists of spherical particles measuring ca. 1 micrometer according to SEM (FIG. 13). The X-ray scattering pattern of the calcined COK-10 material is shown in FIG. 14. The presence of diffraction peaks reveals that the material is ordered at the meso-scale.

### Example 8

### In Vitro Release Experiment of Itraconazole from COK-10 of Example 1

Itraconazole is a poorly soluble drug compound. An amount of 50.00 mg of itraconazole was dissolved in 1 ml of dichloromethane. An amount of 150.03 mg of COK-10 was impregnated with three times 250 μl of the itraconazole solution. The impregnated COK-10 sample was dried in a vacuum oven at 40° C.

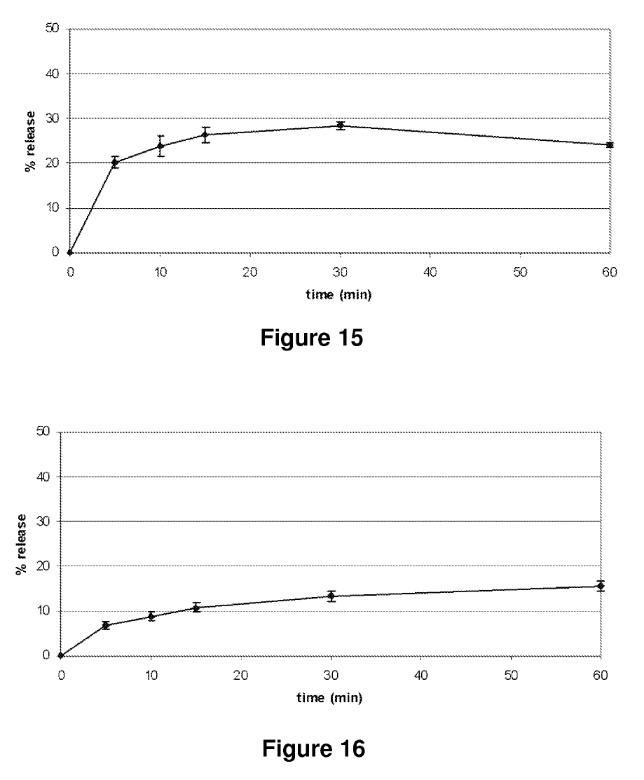

The release medium was simulated gastric fluid (SGF) to which sodium lauryl sulfate (SLS) was added (0.05 wt. %). The itraconazole loaded COK-10 was suspended in 20 ml of dissolution medium. The suspension was agitated at 730 rpm. The loading of the silica materials amounted to 18 wt. %. The concentration of itraconazole in the dissolution bath was determined using HPLC. The release of itraconazole is plotted against time in FIG. 15. In short time the COK-10 formulation releases significant amounts of itraconazole into the dissolution medium. After 5 minutes, 20% of the itraconazole contained in the COK-10 carrier was released. After 30 minutes, the release was close to 30%.

### Example 9

### In Vitro Release Experiment of Itraconazole from a Mesoporous Material not Synthesized According to the Invention (Prepared in Comparative Example 3)

An amount of 49.98 mg of itraconazole was dissolved in 1 ml of dichloromethane. An amount of 150.03 mg of the mesoporous material of Example 3 was impregnated with two times 375 μl of the itraconazole solution. The impregnated mesoporous silica sample was dried in a vacuum oven at 40° C.

The release medium was simulated gastric fluid (SGF) to which sodium lauryl sulfate was added (0.05 wt.-%). The itraconazole loaded mesoporous silica was suspended in 15 ml of dissolution medium. The suspension was agitated at 730 rpm. The loading of the silica carrier with itraconazole amounted to 15.65 wt.-%. The concentration of itraconazole in the dissolution bath was determined using HPLC. The release of itraconazole is plotted against time in FIG. 16. This formulation releases significantly less itraconazole into the dissolution medium compared to the COK-10 sample, cfr. FIG. 15. After 5 minutes, only ca. 7% of the itraconazole was released into the medium. After 60 minutes this amount was increased to 15% only.

### Example 10

### In Vitro Release Experiment of Itraconazole from SBA-15 (Prepared in Comparative Example 4)

An amount of 50.05 mg of itraconazole was dissolved in 1 mL of dichloromethane. An amount of 150.02 mg of the SBA-15 sample prepared as described in Example 4 was impregnated with three times 250 μl of the itraconazole solution. The impregnated SBA-15 sample was dried in a vacuum oven at 40° C.

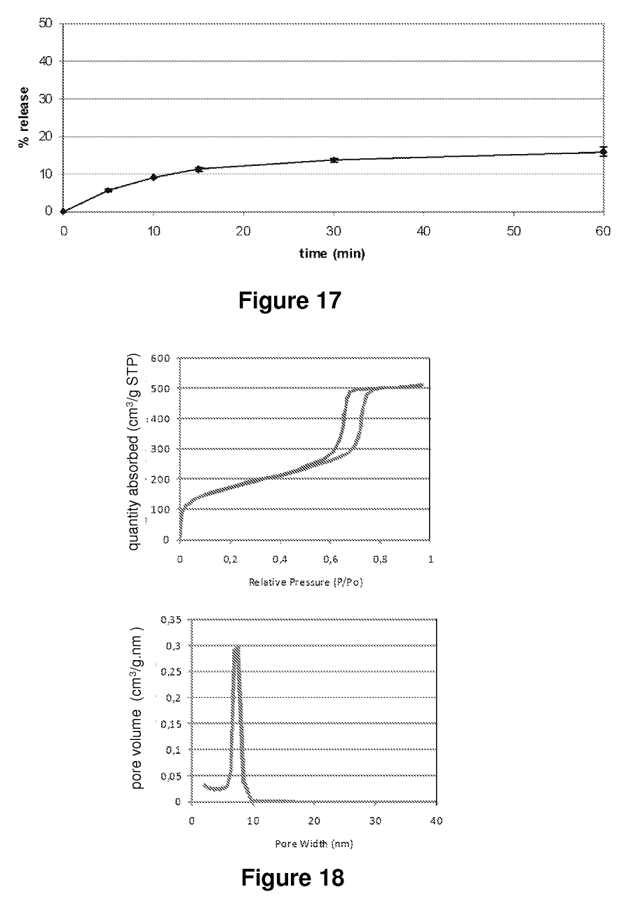

The release medium was simulated gastric fluid (SGF) to which sodium lauryl sulfate was added (0.05 wt. %). The itraconazole loaded mesoporous silica was suspended in 20 ml of dissolution medium. The itraconazole loading of the SBA silica material amounted to 18 wt. %. The suspension was agitated at 1100 rpm. The concentration of itraconazole in the dissolution bath was determined using HPLC. The release of itraconazole is plotted against time in FIG. 17. This formulation releases significantly less itraconazole into the dissolution medium compared to the COK-10 sample, cfr. FIG. 15. After 5 minutes, only ca. 5% of the itraconazole was released from the SBA-15 into the medium. After 60 minutes this amount was increased to ca. 18% only.

### Example 11

### Room Temperature Synthesis of COK-10 Using TPAOH (SiO2/TPAOH=25/1) with the pH of the reaction mixture equal to 6.06

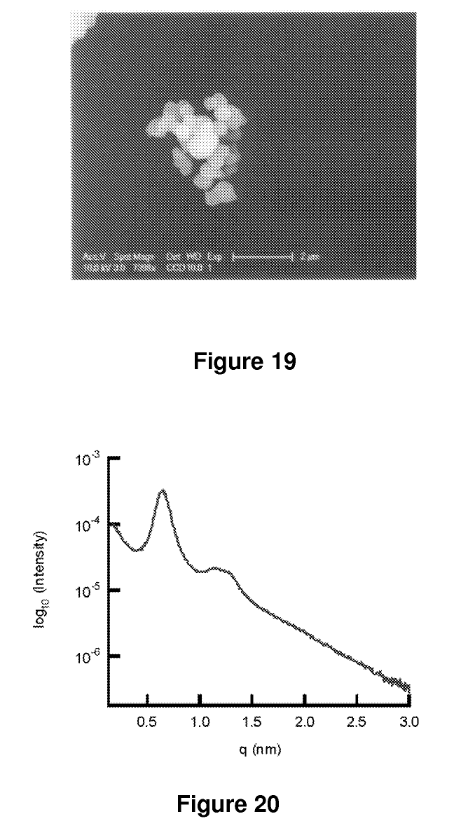

An amount of 4.116 g of Pluronic P123 surfactant was mixed with 107.506 g of water, 12.78 g HCl solution (2.4M) and 1.8 ml of a 1M TPAOH solution in a PP vessel (500 ml). This mixture (mixture 1) was stirred with a magnetic stirrer (400 rpm) at room temperature. In a second PP recipient 10.45 g of a sodium silicate solution was mixed with 30.04 g of water (mixture 2). This mixture was stirred using a magnetic stirrer (400 rpm) at room temperature. The latter solution was added to the surfactant mixture (mixture 1). The resulting reaction mixture is stirred with a direct drive electric mixer (200 rpm) for 5 minutes. At the end of this step the pH was measured to be 6.06 using a Mettler Toledo, InLab®Expert Pro pH electrode and the temperature to be 24° C. The resulting reaction mixture was kept at room temperature for 24 h without stirring. The resulting reaction mixture was vacuum filtered (particle retention 20-25 μm). There was no consequent phase of raising the temperature to 90° C. and holding isothermal for 24 h such in examples 1, 2, 3, 4 and 7. The powder on the filter was washed using 300 ml of water. The resulting powder was dried in a glass recipient for 24 h at 60° C. Finally the powder was transferred to porcelain plates and calcined in an air oven at 550° C. for 8 h using a heating rate of 1° C./min. The nitrogen adsorption isotherm of this sample is shown in FIG. 18 (top). The isotherm shows hysteresis with parallel adsorption and desorption branches, revealing the presence of a uniform pores. The pore diameter is estimated around 8 nm (FIG. 18B, bottom). The particle size and shape was investigated with SEM (FIG. 19). The elementary particle size is around 1 micron. The particles are aggregated into larger bodies (FIG. 19). The X-ray scattering pattern of the calcined material is shown in FIG. 20. The presence of diffraction peaks reveals that the material is ordered at the meso-scale. The performance of this COK-10 mesoporous silica as a carrier for low-soluble drugs was evaluated in an in vitro release experiment of itraconazole. The mesoporous carrier was loaded with 21.38 wt % itraconazole. In short time the COK-10 formulation releases significant amounts of itraconazole into the dissolution medium.

### Example 12

### Room Temperature Synthesis of COK-10 Using TMAOH (SiO2/TPAOH=25/1) with the pH of the Reaction Mixture Equal to 5.75

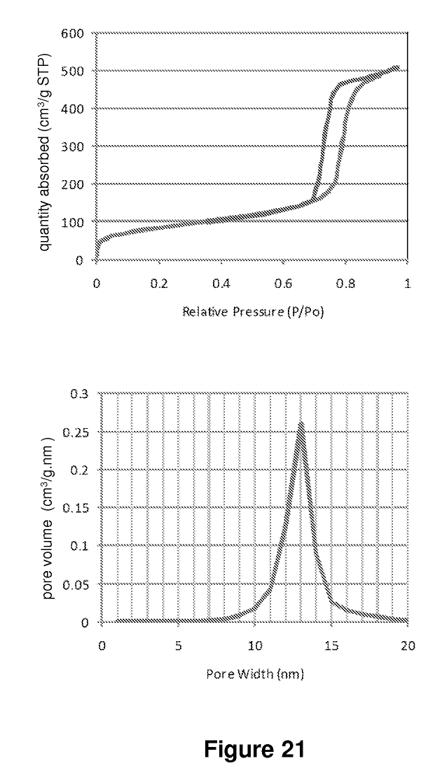

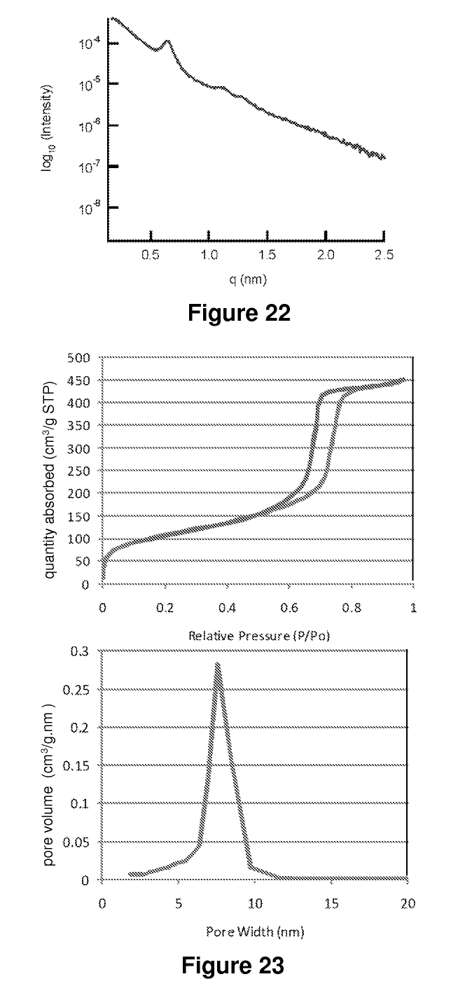

An amount of 4.154 g of Pluronic P123 surfactant was mixed with 107.606 g of water, 12.762 g HCl solution (2.4M) and 1.8 ml of a 1M TMAOH solution in a PP vessel (500 ml). This mixture (mixture 1) was stirred with a magnetic stirrer (400 rpm) at room temperature. In a second PP recipient 10.463 g of a sodium silicate solution was mixed with 30.03 g of water (mixture 2). This mixture was stirred using a magnetic stirrer (400 rpm) at room temperature. The latter solution was added to the surfactant mixture (mixture 1). The resulting reaction mixture is stirred with a direct drive electric mixer (200 rpm) for 5 minutes. At the end of this step the pH was measured to be 5.75 using a Mettler Toledo, InLab®Expert Pro pH electrode and the temperature to be 22° C. The resulting reaction mixture was kept at room temperature for 24 h without stirring. After 24 h the reaction mixture was placed in an oven at 90° C. for 24 h. The resulting reaction mixture was vacuum filtered (particle retention 20-25 μm). The powder on the filter was washed using 300 ml of water. The resulting powder was dried in a glass recipient for 24 h at 60° C. Finally the powder was transferred to porcelain plates and calcined in an air oven at 550° C. for 8 h using a heating rate of 1° C./min. The nitrogen adsorption isotherm of this sample is shown in FIG. 21 (top). The isotherm shows hysteresis with parallel adsorption and desorption branches, revealing the presence of a uniform pores. The pore diameter is estimated around 12 nm (FIG. 21B, bottom). The X-ray scattering pattern of the calcined COK-10 material is shown in FIG. 22. The presence of diffraction peaks reveals that the material is ordered at the meso-scale.

### Example 13

### Synthesis of COK-10 with the pH of the Reaction Mixture Equal to 6.5

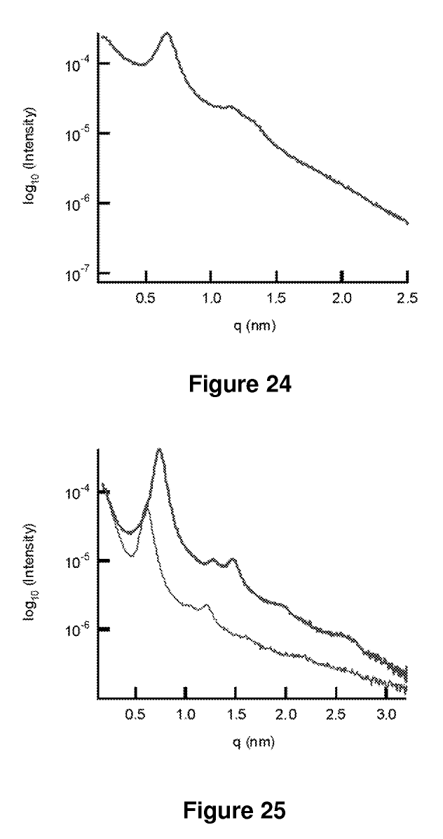

An amount of 4.090 g of Pluronic P123 surfactant was mixed with 107.544 g of water, 12.017 g HCl solution (2.4M) in a PP vessel (500 ml). This mixture (mixture 1) was stirred with a magnetic stirrer (400 rpm) at room temperature. In a second PP recipient 10.43 g of a sodium silicate solution was mixed with 31.0 g of water (mixture 2). This mixture was stirred using a magnetic stirrer (400 rpm) at room temperature. The latter solution was added to the surfactant mixture (mixture 1). The resulting reaction mixture is stirred with a direct drive electric mixer (200 rpm) for 5 minutes. At the end of this step the pH was measured to be 6.5 using a Mettler Toledo, InLab®Expert Pro pH electrode and the temperature to be 22° C. The resulting reaction mixture was kept at room temperature for 24 h without stirring. There was no consequent phase of raising the temperature to 90° C. and holding isothermal for 24 h. The resulting reaction mixture was vacuum filtered (particle retention 20-25 μm). The powder on the filter was washed using 300 ml of water. The resulting powder was dried in a glass recipient for 24 h at 60° C. Finally the powder was transferred to porcelain plates and calcined in an air oven at 550° C. for 8 h using a heating rate of 1° C./min. The nitrogen adsorption isotherm of this sample is shown in FIG. 23 (top). The isotherm shows hysteresis with parallel adsorption and desorption branches, revealing the presence of a uniform pores. The pore diameter is estimated around 8 nm (FIG. 23B, bottom). The X-ray scattering pattern of the calcined COK-10 material is shown in FIG. 24. The presence of diffraction peaks reveals that the material is ordered at the meso-scale.

### Example 14

### Buffer Mediated Synthesis of COK-12 (Ordered Mesoporous Material) at Room Temperature with the pH of the Reaction Mixture Equal to 5.2

An amount of 4.060 g of Pluronic P123 surfactant was mixed with 107.672 g of water, 2.87 g Sodium Citrate and 3.41 g Citric Acid in a PP vessel (500 ml). This solution was stirred (400 rpm) overnight with a magnetic stirring bar. The pH of the solution was equal to 3.8 and the temperature 22° C. (Mettler Toledo, InLab®Expert Pro pH electrode).

In a PP beaker (50 ml) 10.420 g of a sodium silicate solution (Riedel-de Haën, purum, ≧10% NaOH basis, 27% SiO2 basis) was mixed with 30.012 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 5.2 after 3 minutes. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

The X-ray scattering pattern of the as-synthesized and calcined material is shown in FIG. 25. The material is ordered at the meso-scale with a 2D-hexagonal structure (p6m space group). The unit cell parameter a is equal to 9.872 nm.

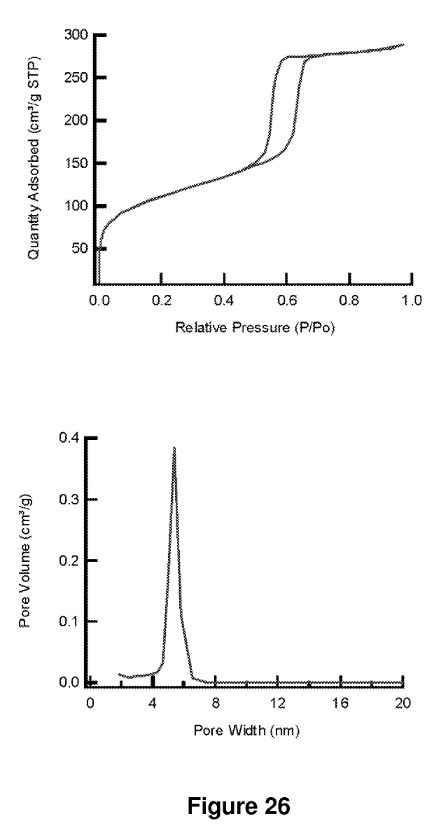

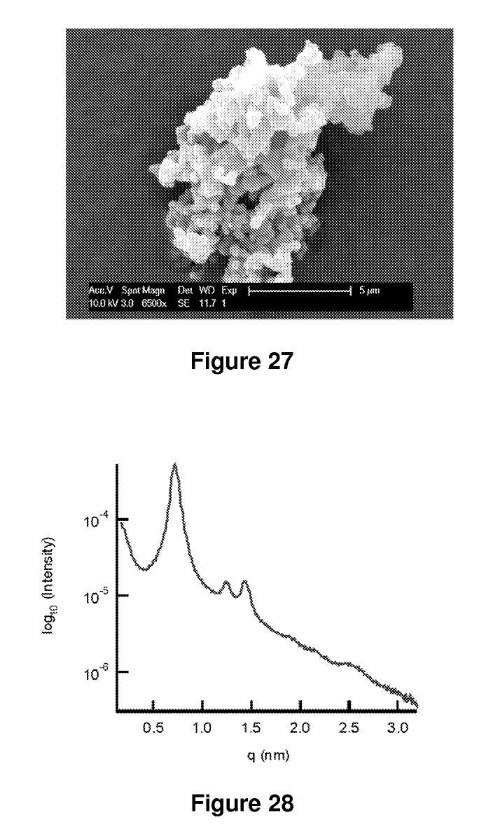

The nitrogen adsorption isotherm of the calcined COK-12 material is shown in FIG. 26 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 26B, bottom). The pore size is ca. 5 nm. The results from nitrogen adsorption (FIG. 26) together with X-ray scattering (FIG. 25) show that this sample is an ordered mesoporous material. The morphology of the sample was investigated with SEM (FIG. 27). The material consists of a network of intergrown particles.

### Example 15

### Buffer Mediated Synthesis of COK-12 at Room Temperature with the pH of the Reaction Mixture Equal to 4.9

An amount of 4.109 g of Pluronic P123 surfactant was mixed with 107.573 g of water, 2.540 g Sodium Citrate and 3.684 g Citric Acid in a PP vessel (500 ml). This solution was stirred (400 rpm) overnight with a magnetic stirring bar. The pH of the solution was equal to 3.6 and the temperature 22° C. (Mettler Toledo, InLab®Expert Pro pH electrode).

In a PP beaker (50 ml) 10.424 g of a sodium silicate solution (Riedel-de Haën, purum, ≧10% NaOH basis, ≧27% SiO2 basis) was mixed with 30.091 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 4.9 after 3 minutes. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

The X-ray scattering pattern of the as-synthesized and calcined COK-12 material is shown in FIG. 28. The material is ordered at the meso-scale with a 2D-hexagonal structure (p6m space group). The unit cell parameter a is equal to 10.091 nm.

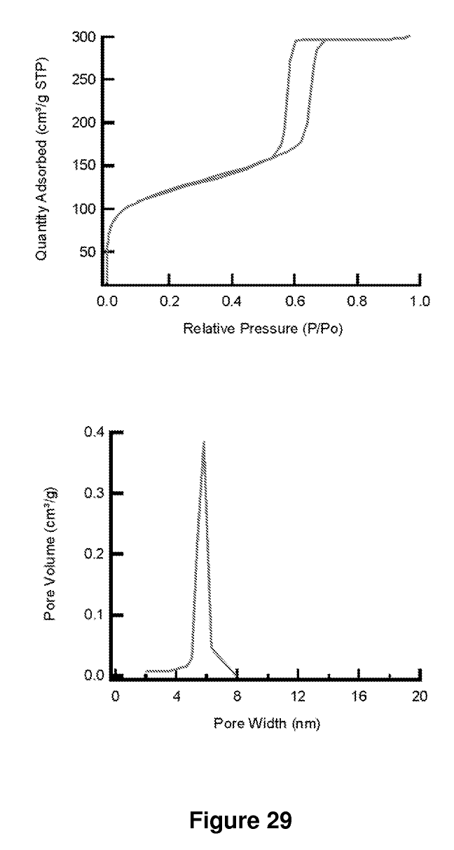

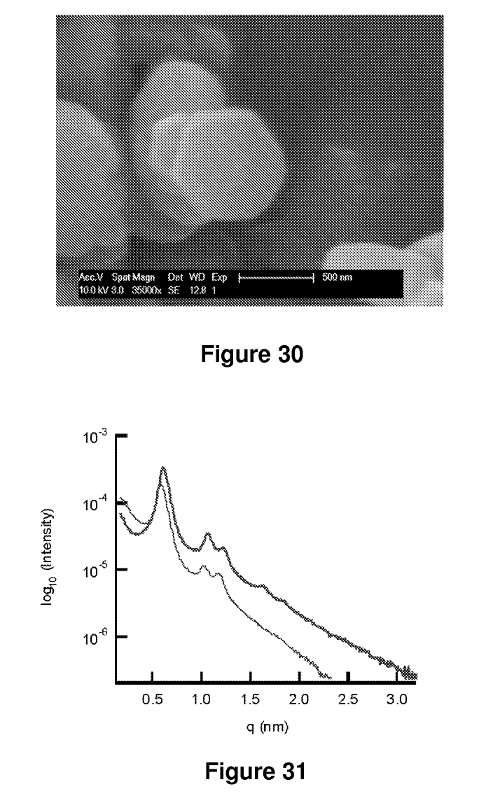

²⁹Si MAS NMR spectra of the as-synthesized material was recorded on a Bruker AMX300 spectrometer (7.0 T). 4000 scans were accumulated with a recycle delay of 60 s. The sample was packed in a 4 mm Zirconia rotor. The spinning frequency of the rotor was 5000 Hz. Tetramethylsilane was used as shift reference. The Q3 and Q4 silica species were observed as broad peaks at −99 and −109 ppm respectively with a Q3/Q4 ratio equal to 0.59 was found implying that the silica walls of this COK-12 material are highly condensed. This value can be compared with the Q3/Q4 ratio (0.78) of SBA-15 samples (Zhao et al., J. Am. Chem. Soc., 1998, Vol 120, No. 24, p6024). The nitrogen adsorption isotherm of the calcined COK-12 material is shown in FIG. 29 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 29B, bottom). The pore size is ca. 5 nm. The results from nitrogen adsorption (FIG. 29) together with X-ray scattering (FIG. 28) show that this sample is an ordered mesoporous material. The morphology of the sample was investigated with SEM (FIG. 30). The material consists of a network of intergrown particles.

### Example 16

### Buffer Mediated Synthesis of COK-12 at 90° C. with the pH of the Reaction Mixture Equal to 4.6

An amount of 4.116 g of Pluronic P123 surfactant was mixed with 107.495 g of water, 5.104 g Sodium Citrate and 4.335 g Citric Acid in a PP vessel (500 ml). This solution was stirred (400 rpm) overnight with a magnetic stirring bar. The pH of the solution was equal to 3.8 and the temperature 22° C. (Mettler Toledo, InLab®Expert Pro pH electrode).

In a PP beaker (50 ml) 10.434 g of a sodium silicate solution (Riedel-de Haën, purum, ≧10% NaOH basis, ≧27% SiO2 basis) was mixed with 30.586 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 4.6 after 3 minutes. The bottle was kept at room temperature for 24 h and 24 h at 90° C. in an oven. The resulting reaction mixture was cooled down to RT and vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

The X-ray scattering pattern of the as-synthesized and calcined material is shown in FIG. 31. The material is ordered at the meso-scale with a 2D-hexagonal structure (p6m space group). The unit cell parameter a is equal to 11.874.

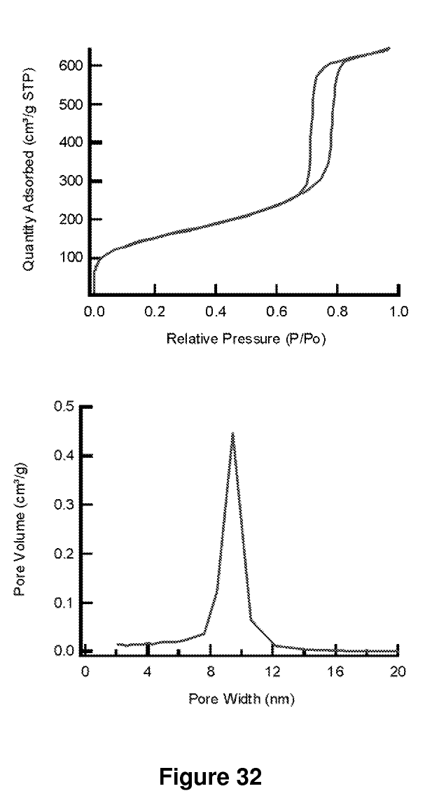

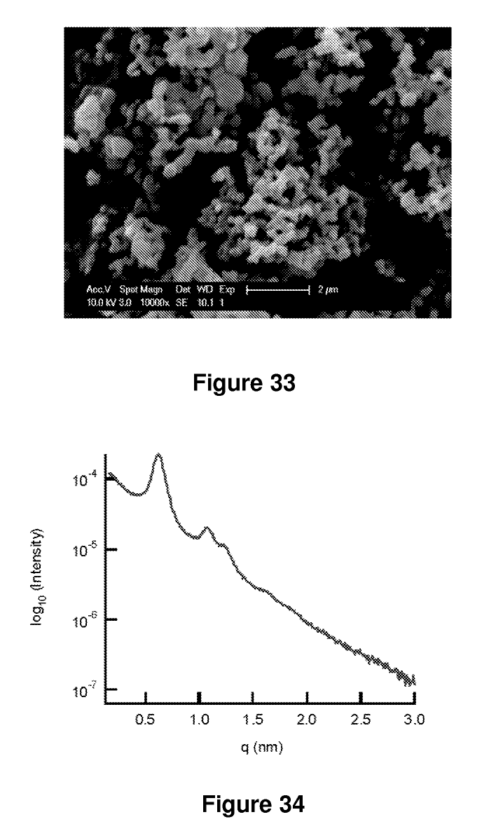

The nitrogen adsorption isotherm of the calcined COK-12 material is shown in FIG. 32 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 32B, bottom). The pore size is ca. 10 nm. The results from nitrogen adsorption (FIG. 32) together with X-ray scattering (FIG. 31) show that this sample is an ordered mesoporous material. The morphology of the sample was investigated with SEM (FIG. 33). The material consists of a network of intergrown particles.

### Example 17

### Buffer Mediated Synthesis of COK-12 at 90° C. with the pH of the Reaction Mixture Equal to 5.6

An amount of 4.140 g of Pluronic P123 surfactant was mixed with 107.574 g of water, 7.340 g Sodium Citrate and 3.005 g Citric Acid in a PP vessel (500 ml). This solution was stirred (400 rpm) overnight with a magnetic stirring bar. The pH of the solution was equal to 4.7 and the temperature 22° C. (Mettler Toledo, InLab®Expert Pro pH electrode).

In a PP beaker (50 ml) 10.405 g of a sodium silicate solution (Riedel-de Haën, purum, ≧10% NaOH basis, ≧27% SiO2 basis) was mixed with 30.578 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 5.6 after 3 minutes. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

The X-ray scattering pattern of the as-synthesized and calcined COK-12 material is shown in FIG. 34. The material is ordered at the meso-scale with a 2D-hexagonal structure (p6m space group). The unit cell parameter a is equal to 11.721 nm.

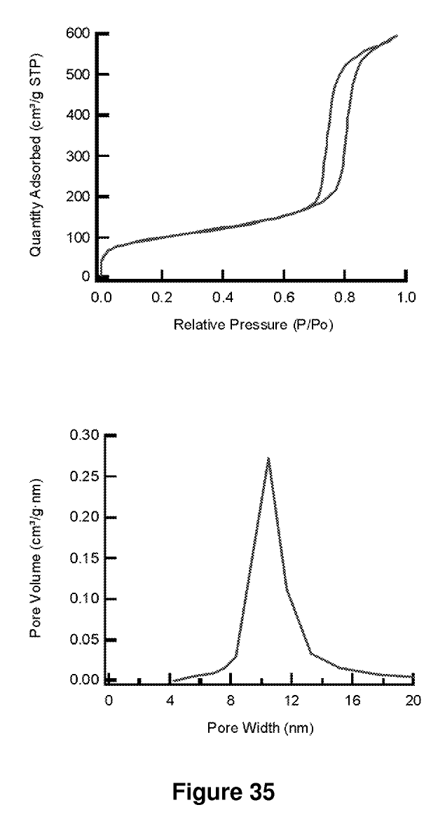

The nitrogen adsorption isotherm of the calcined COK-12 material is shown in FIG. 35 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 35B, bottom). The pore size is ca. 11 nm. The results from nitrogen adsorption (FIG. 35) together with X-ray scattering (FIG. 34) show that this sample is an ordered mesoporous material.

### Example 18

### Buffer Mediated Synthesis of COK-12 at Room Temperature with the pH of the Reaction Mixture Equal to 6.0

An amount of 4.069 g of Pluronic P123 surfactant was mixed with 107.524 g of water, 7.993 g Sodium Citrate and 2.461 g Citric Acid in a PP vessel (500 ml). This solution was stirred (400 rpm) overnight with a magnetic stirring bar. The pH of the solution was equal to 4.9 and the temperature 22° C. (Mettler Toledo, InLab®Expert Pro pH electrode).

In a PP beaker (50 ml) 10.400 g of a sodium silicate solution (Riedel-de Haën, purum, ≧10% NaOH basis, ≧27% SiO2 basis) was mixed with 30.000 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 6.0 after 3 minutes. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

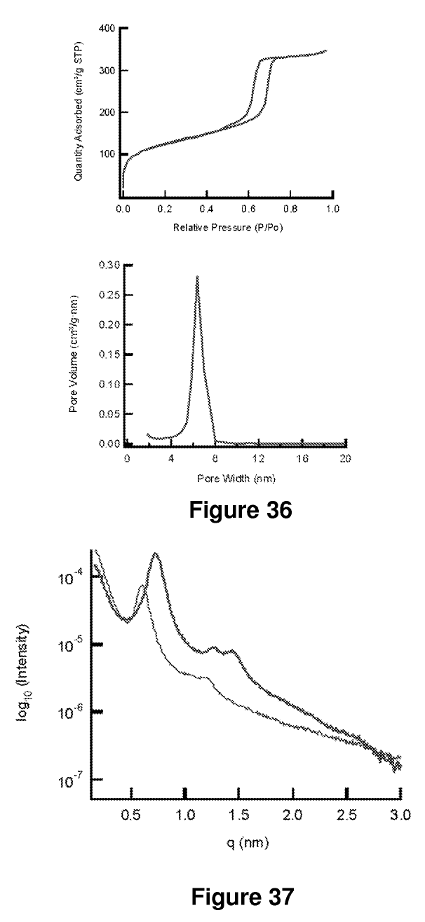

The nitrogen adsorption isotherm of the calcined COK-12 material is shown in FIG. 36 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 36B, bottom). The pore size is ca. 5 nm.

### Example 19

### Buffer Mediated Synthesis of COK-12 at Room Temperature with the pH of the Reaction Mixture Equal to 5.6

An amount of 4.087 g of Pluronic P123 surfactant was mixed with 107.625 g of water, 7.308 g Sodium Citrate and 2.994 g Citric Acid in a PP vessel (500 ml). This solution was stirred (400 rpm) overnight with a magnetic stirring bar. The pH of the solution was equal to 4.7 and the temperature 22° C. (Mettler Toledo, InLab®Expert Pro pH electrode).

In a PP beaker (50 ml) 10.410 g of a sodium silicate solution (Riedel-de Haën, purum, ≧10% NaOH basis, 27% SiO2 basis) was mixed with 30.040 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 5.6 after 3 minutes. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

The X-ray scattering pattern of the as-synthesized and calcined material is shown in FIG. 37. The material is ordered at the meso-scale with a 2D-hexagonal structure (p6m space group). The unit cell parameter a is equal to 9.980 nm.

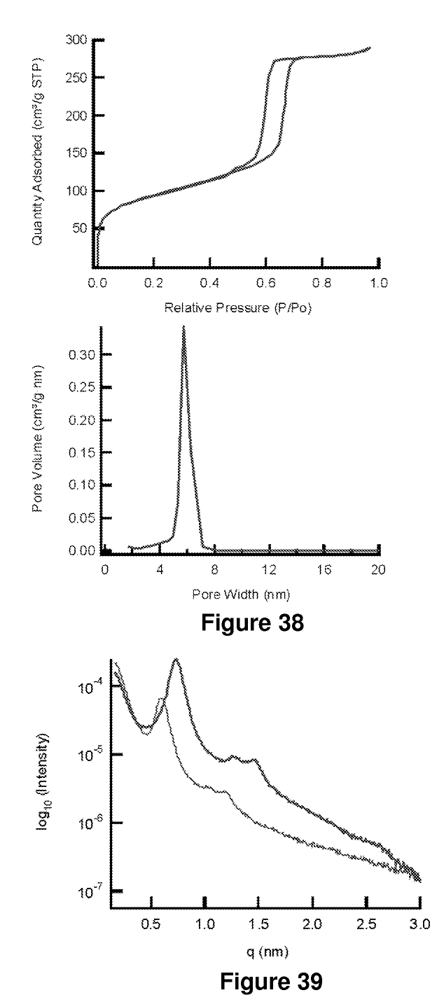

The nitrogen adsorption isotherm of the calcined COK-12 material is shown in FIG. 38 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 38B, bottom). The pore size is ca. 5 nm. The results from nitrogen adsorption (FIG. 38) together with X-ray scattering (FIG. 37) show that this sample is an ordered mesoporous material.

### Example 20

### Buffer Mediated Synthesis of COK-12 at Room Temperature with the pH of the Reaction Mixture Equal to 5.3

An amount of 4.142 g of Pluronic P123 surfactant was mixed with 107.817 g of water, 6.542 g Sodium Citrate and 3.674 g Citric Acid in a PP vessel (500 ml). This solution was stirred (400 rpm) overnight with a magnetic stirring bar. The pH of the solution was equal to 4.4 and the temperature 22° C. (Mettler Toledo, InLab®Expert Pro pH electrode).

In a PP beaker (50 ml) 10.400 g of a sodium silicate solution (Riedel-de Haën, purum, ≧10% NaOH basis, ≧27% SiO2 basis) was mixed with 30.10 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 5.3 after 3 minutes. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

The X-ray scattering pattern of the as-synthesized and calcined material is shown in FIG. 39. The material is ordered at the meso-scale with a 2D-hexagonal structure (p6m space group). The unit cell parameter a is equal to 9.871 nm.

The nitrogen adsorption isotherm of the calcined COK-12 material is shown in FIG. 40 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 40B, bottom). The pore size is ca. 5 nm. The results from nitrogen adsorption (FIG. 40) together with X-ray scattering (FIG. 39) show that this sample is an ordered mesoporous material.

### Example 21

### Buffer Mediated Synthesis of COK-12 at Room Temperature with the pH of the Reaction Mixture Equal to 5.1

An amount of 4.149 g of Pluronic P123 surfactant was mixed with 107.523 g of water, 5.771 g Sodium Citrate and 4.086 g Citric Acid in a PP vessel (500 ml). This solution was stirred (400 rpm) overnight with a magnetic stirring bar. The pH of the solution was equal to 4.2 and the temperature 22° C. (Mettler Toledo, InLab®Expert Pro pH electrode).

In a PP beaker (50 ml) 10.409 g of a sodium silicate solution (Riedel-de Haën, purum, ≧10% NaOH basis, ≧27% SiO2 basis) was mixed with 30.032 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 5.1 after 3 minutes. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

The X-ray scattering pattern of the as-synthesized and calcined material is shown in FIG. 41. The material is ordered at the meso-scale with a 2D-hexagonal structure (p6m space group). The unit cell parameter a is equal to 9.980 nm.

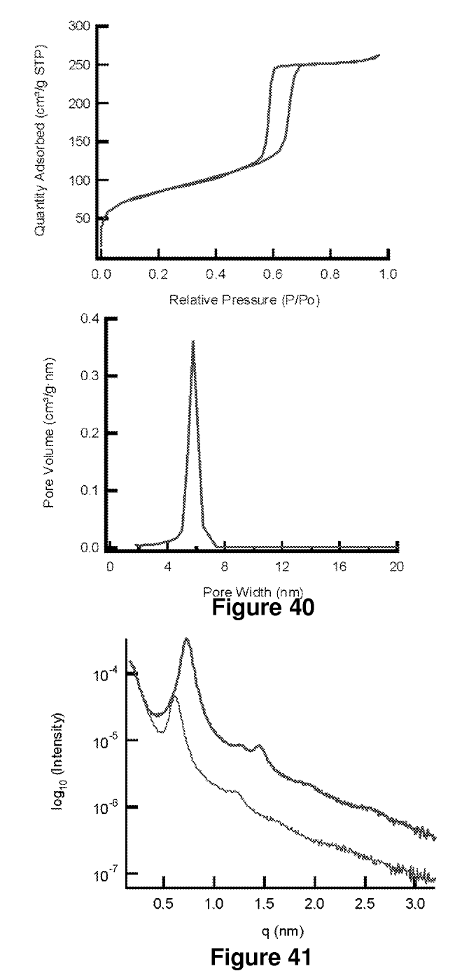

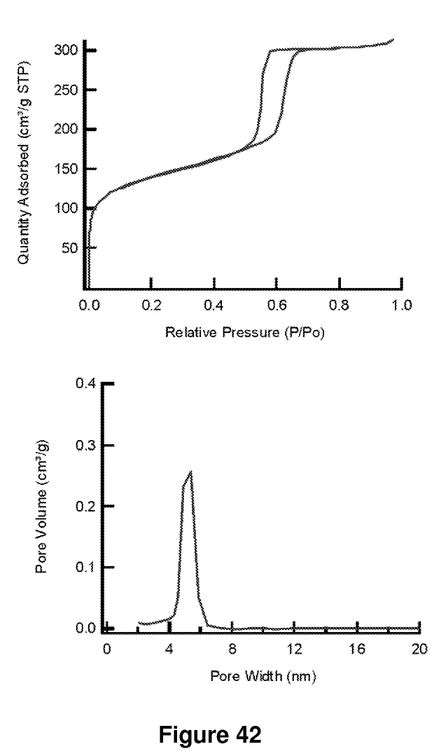

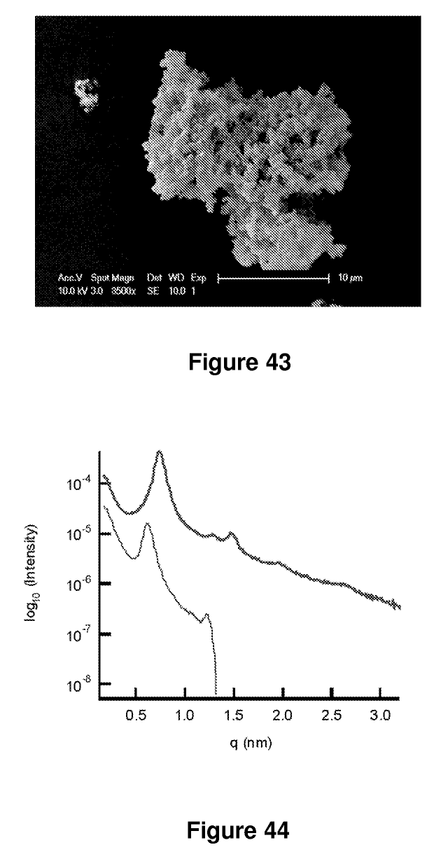

The nitrogen adsorption isotherm of the calcined COK-12 material is shown in FIG. 42 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 42B, bottom). The pore size is ca. 5 nm. The results from nitrogen adsorption (FIG. 42) together with X-ray scattering (FIG. 43) shows that this sample is an ordered mesoporous material. The morphology of the sample was investigated with SEM (FIG. 43). The material consists of a network of intergrown particles.

### Example 22

### Buffer Mediated Synthesis of COK-12 at Room Temperature with the pH of the Reaction Mixture Equal to 4.6

An amount of 4.129 g of Pluronic P123 surfactant was mixed with 107.520 g of water, 5.771 g Sodium Citrate and 4.086 g Citric Acid in a PP vessel (500 ml). This solution was stirred (400 rpm) overnight with a magnetic stirring bar. The pH of the solution was equal to 3.8 and the temperature 22° C. (Mettler Toledo, InLab®Expert Pro pH electrode).

In a PP beaker (50 ml) 10.409 g of a sodium silicate solution (Riedel-de Haën, purum, ≧10% NaOH basis, ≧27% SiO₂ basis) was mixed with 30.032 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 4.6 after 3 minutes. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

The X-ray scattering pattern of the as-synthesized and calcined material is shown in FIG. 44. The material is ordered at the meso-scale with a 2D-hexagonal structure (p6m space group). The unit cell parameter a is equal to 9.765 nm.

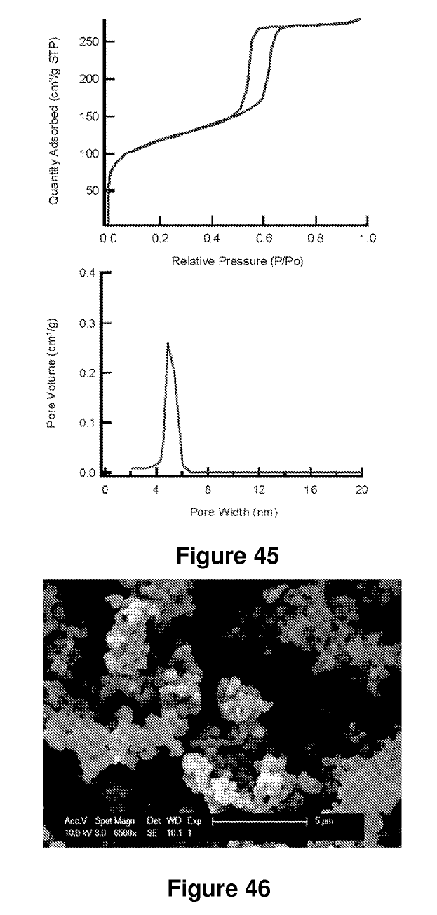

The nitrogen adsorption isotherm of the calcined COK-12 material is shown in FIG. 45 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 45B, bottom). The pore size is ca. 5 nm. The results from nitrogen adsorption (FIG. 45) together with X-ray scattering (FIG. 44) show that this sample is an ordered mesoporous material. The morphology of the sample was investigated with SEM (FIG. 46). The material consists of a network of intergrown particles.

### Example 23

### Buffer Mediated Synthesis of COK-12 at Room Temperature with the pH of the Reaction Mixture Equal to 3.5

An amount of 4.074 g of Pluronic P123 surfactant was mixed with 108.436 g of water, 0.751 g Sodium Citrate and 7.695 g Citric Acid in a PP vessel (500 ml). This solution was stirred (400 rpm) overnight with a magnetic stirring bar. The pH of the solution was equal to 3.5 and the temperature 22° C. (Mettler Toledo, InLab®Expert Pro pH electrode).

In a PP beaker (50 ml) 10.414 g of a sodium silicate solution (Riedel-de Haën, purum, ≧10% NaOH basis, ≧27% SiO₂ basis) was mixed with 30.059 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 3.5 after 3 minutes. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 550° C. for 8 h using a heating rate of 1° C./min.

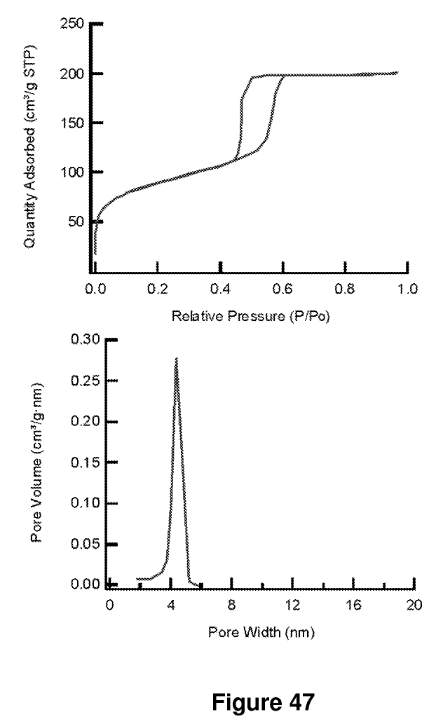

The nitrogen adsorption isotherm of the calcined COK-12 material is shown in FIG. 47 (top). The type IV isotherm is characteristic of a mesoporous material. The steep parallel branches of the hysteresis loop indicate that the pore sizes are quite uniform. The pore size distribution was derived from the nitrogen adsorption isotherm using the BJH method (FIG. 47B, bottom). The pore size is ca. 4.5 nm.

### Example 24

### Synthesis of COK-12 with In Situ Buffer Formation at Room Temperature with the pH of the Reaction Mixture Equal to 5.20 (Without Addition of Sodium Citrate)

An amount of 4.00 g of Pluronic P123 surfactant was mixed with 107.50 g of water and 2.79 g Citric Acid in a PP vessel (500 ml). This solution was stirred (400 rpm) overnight with a magnetic stirring bar. The pH of the solution was equal to 1.90 and the temperature 22° C. (Mettler Toledo, InLab®Expert Pro pH electrode).

In a PP beaker (50 ml) 10.42 g of a sodium silicate solution (Merck 8% Na₂O, 27% SiO2 basis) was mixed with 30.01 g of water. This mixture was stirred using a magnetic stirrer (400 rpm) at room temperature for 5 minutes. The latter solution was added to the surfactant solution in the PP bottle under mechanical stirring (200 rpm). The resulting solution is stirred (200 rpm) for 5 minutes at RT. The pH stabilized at 5.20 after 0.5 minute. The bottle was kept at room temperature for 24 h. The resulting reaction mixture was vacuum filtered (particle retention 20-25 μm). The powder on the filter was washed using 300 ml of water. The resulting powder was dried in a glass recipient for 24 h at 60° C. The as-synthesized powder was transferred to porcelain plates and calcined in an air oven at 300° C. for 8 h and another 8 h at 550° C. using a heating rate of 1° C./min.

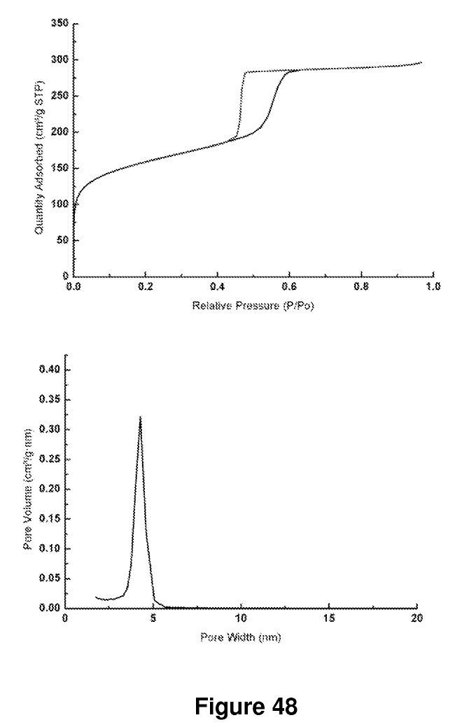

The nitrogen adsorption isotherm of the calcined COK-12 material is shown in FIG. 48 (top). The type IV isotherm is characteristic of a mesoporous material. The pore size distribution is narrow with a mean diameter of 4.3 nm (see FIG. 48B, bottom). The following examples teach the synthesis of COK-12 and illustrate the most favorable synthesis conditions for obtaining a narrow mesopore size distribution.

### Example 25

Aqueous (surfactant P123) solution 1 was prepared by dissolving 10 g P123 (BASF, Belgium), 9.2 g citric Acid monohydrate (Riedel-de Haen, Germany) and 6.35 g trisodium citrate (UCB, Belgium) in 268.75 g of deionized water with a pH of 4.0

Aqueous (silicate precursor) solution 2 was prepared by diluting 4.83 g of sodium silicate solution (extra pure, 7.5 wt % Na₂O and 26.5-28.5 wt % SiO₂ from Merck, Germany) with 13.95 g of deionized water with a pH of 11.0.

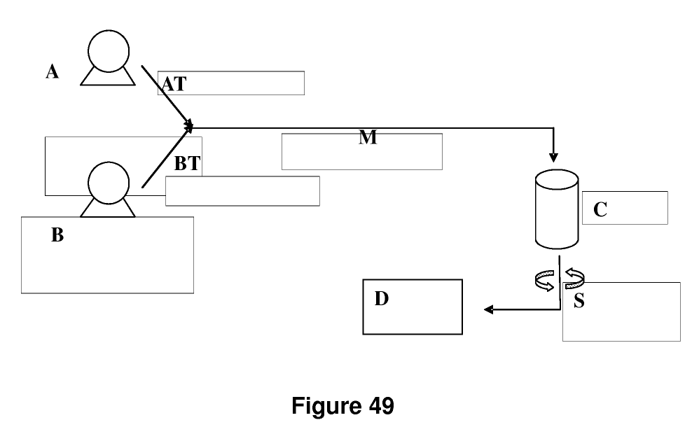

The experimental setup used is shown schematically in FIG. 49 in which A is the aqueous solution A comprising a silica precursor; AT is the tube transporting the aqueous solution A; B is the aqueous solution B comprising a poly(alkylene oxide) triblock copolymer and an acid with a pKa in the range 3 to 9; BT is the tube transporting the aqueous solution B; M is the mixing tube; C is the collector; S is the separator; and D is the drier/calcinating oven. The two syringes of the perfusion pumps were each 20 cm long, with a 1.6 mm internal diameter and cylindrical ends and were connected by a Swagelok T-coupling piece under a 90° angle to a 160 cm long mixing tube with an internal diameter of 1.6 mm. The two syringes were filled with aqueous solution 1 and aqueous solution 2 respectively and aqueous solution 1 and aqueous solution 2 were jetted into one another at rates of 99.0 mL/hour (49.2 m/h) and 37.2 mL/hr (18.5 m/h) respectively into the 160 cm long mixing tube realizing a pH of 5.0 in the mixture. The linear velocity through the mixing pipe was 67.77 m/h and the residence time of the mixture of aqueous solution 1 and aqueous solution 2 in the mixing tube was 85 s.

The pumps started simultaneously. Upon contact silica condensation was readily visible. Particles flowed out through the tubing and were collected in vials. The particles recovered through centrifugation and decantation were washed 3 times with deionized water. The resulting material was first dried at 100° C. for 2 h and then calcined in an oven by heating up at a rate of 1° C./min to a temperature of 300° C. and then holding the temperature at 300° C. for 8 h.

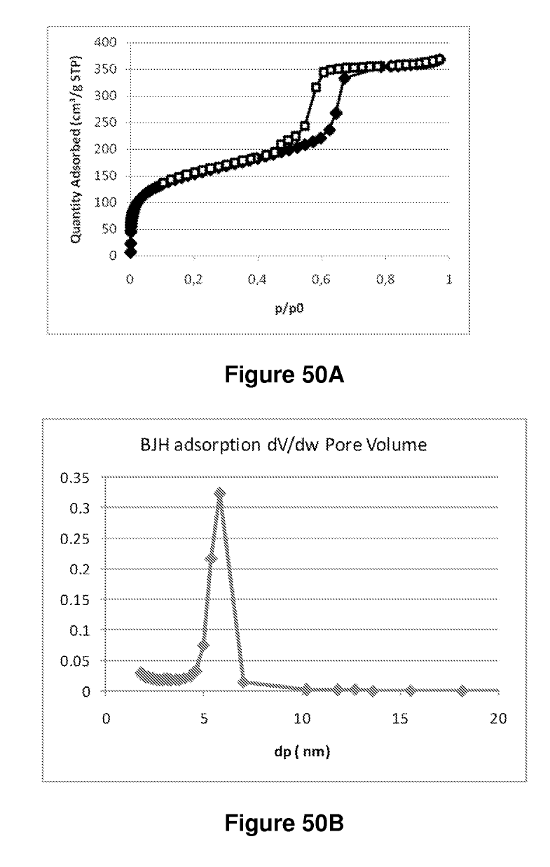

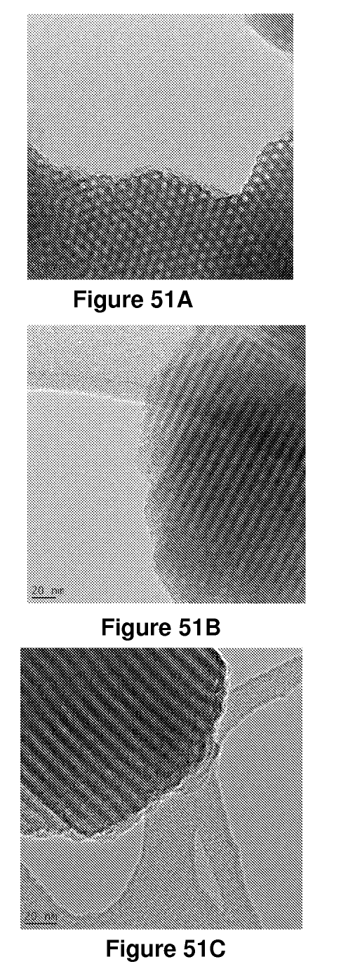

The resulting material was characterized by nitrogen adsorption performed on a Micromeritics Tristar apparatus. Prior to measurement, the sample was pretreated at 200° C. for 10 h (ramp: 5° C./min). The isotherm and pore size distribution are shown in FIGS. 50A and 50B respectively and the P6m hexagonal ordering of the mesophase is clearly illustrated in the TEM micrographs of the resulting calcined mesoporous silica shown in FIGS. 51A, 51B and 51C.

### Example 26

Aqueous (surfactant P123) solution 3 was prepared by dissolving 28.6 g P123 (BASF, Belgium), 25.8 g citric Acid monohydrate (Riedel-de Haen, Germany) and 17.8 g trisodium citrate (UCB, Belgium) in 753.8 g of deionized water with a pH of 4.0.

Aqueous (silicate precursor) solution 4 was prepared by diluting 7.27 g of sodium silicate solution (extra pure, 7.5 wt % Na₂O and 26.5-28.5 wt % SiO₂ from Merck, Germany) with 20.98 g of deionized water with a pH of 11.0.

A similar experimental set-up was used as for Example 1 except that the mixing tube was shorter (30 cm long) and had a larger internal diameter. The two syringes of the perfusion pumps each 20 cm long and with a 1.6 mm internal diameter were filled with aqueous solution 3 and aqueous solution 4 respectively and aqueous solution 3 and aqueous solution 4 were jetted into one another at rates of 198 mL/hour (98.5 m/h) and 74.4 mL/hr (37.0 m/h) respectively into a 30 cm long mixing tube with an internal diameter of 3.2 mm realizing a pH of 5.6 in the mixture. The linear velocity through the mixing pipe was 33.89 m/h and the residence time of the mixture of aqueous solution 3 and aqueous solution 4 in the mixing tube was 31.9 s.

The pumps started simultaneously. Upon contact silica condensation was readily visible. Particles flowed out through the tubing and were collected in vials. The particles recovered through vacuum filtration and were washed with 300 mL of deionized water. The resulting material was first dried at 100° C. for 2 h and then calcined in an oven by heating up at a rate of 1° C./min to a temperature of 550° C. and then holding the temperature at 550° C. for 8 h.

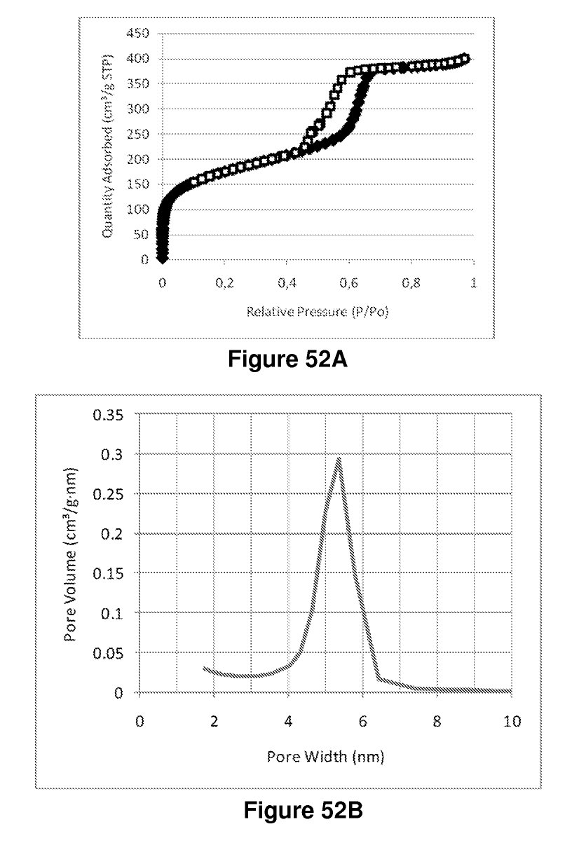

The resulting material was characterized by nitrogen adsorption performed on a Micromeritics Tristar apparatus. Prior to measurement, the sample was pretreated at 300° C. for 10 h (ramp: 5° C./min). The isotherm and pore size distribution are shown in FIGS. 52A and 52B respectively.

### Example 27

Aqueous (surfactant P123) solution 5 was prepared by dissolving 10.10 g P123 (BASF, Belgium), 7.33 g citric Acid monohydrate (Riedel-de Haen, Germany) and 17.8 g trisodium citrate (UCB, Belgium) in 263.41 g of deionized water with a pH of 4.7.

Aqueous (silicate precursor) solution 6 was prepared by diluting 11.68 g of sodium silicate solution (extra pure, 7.5 wt % Na₂O and 26.5-28.5 wt % SiO₂ from Merck, Germany) with 33.74 g of deionized water with a pH of 11.0.

The same experimental setup was used as for Example 2. The two syringes of the perfusion pumps each 20 cm long and with a 1.6 mm internal diameter were filled with aqueous solution 5 and aqueous solution 6 respectively and aqueous solution 5 and aqueous solution 6 were jetted into one another at rates of 198.0 mL/hour (98.5 m/h) and 74.4 mL/hr (37.0 m/h) respectively into a 30 cm long mixing tube with an internal diameter of 3.2 mm realizing a pH of 5.6 in the mixture. The linear velocity through the mixing pipe was 33.89 m/h and the residence time of the mixture of aqueous solution 5 and aqueous solution 6 in the mixing tube was 31.9 s.

The pumps started simultaneously. Upon contact silica condensation was readily visible. Particles flowed out through the tubing and were collected in vials. The particles recovered through vacuum filtration and were washed with deionized water. The resulting material was first dried at 100° C. for 2 h, then calcined in an oven by heating up at a rate of 1° C./min to a temperature of 300° C. and then holding the temperature at 300° C. for 8 h and then heating up at a rate of 1° C./min to a temperature of 550° C. for 8 hours.

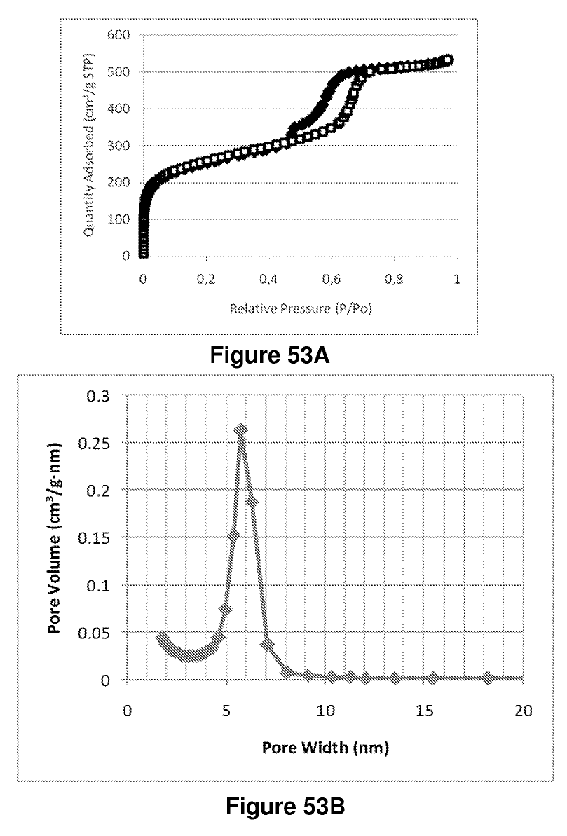

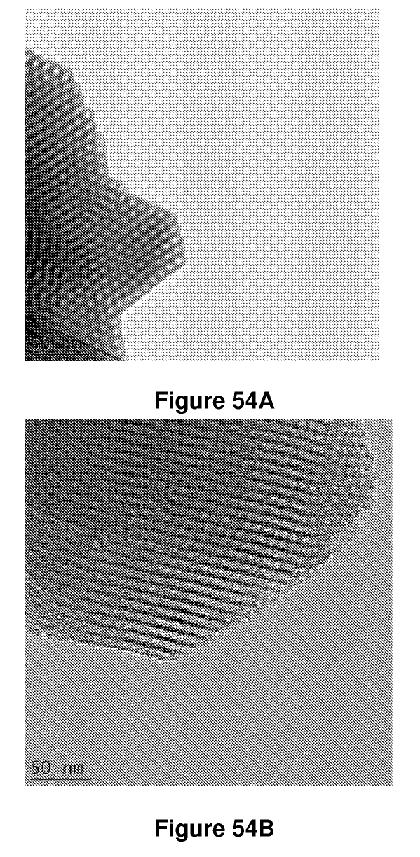

The resulting material was characterized by nitrogen adsorption performed on a Micromeritics Tristar apparatus. Prior to measurement, the sample was pretreated at 300° C. for 10 h (ramp: 5° C./min). The isotherm and pore size distribution are shown in FIGS. 53A and 53B respectively and the P6m hexagonal ordering of the mesophase is clearly illustrated in the TEM micrographs of the resulting calcined mesoporous silica shown in FIGS. 54A and 54B.

### Example 28

Aqueous (silicate precursor) solution 8 was prepared by diluting 145.28 g of sodium silicate solution (extra pure, 7.5 wt % Na₂O and 26.5-28.5 wt % SiO₂ from Merck, Germany) with 419.65 g of deionized water with a pH of 11.0.

Aqueous (surfactant P123) solution 9 was prepared by dissolving 4 g P123 (BASF, Belgium) overnight in an aqueous buffer solution consisting of 3.7 g citric Acid monohydrate (Riedel-de Haen, Germany) and 2.54 g trisodium citrate (UCB, Belgium) in 107.5 g of deionized water with a pH of 4.9.

The experimental setup used is shown in schematically in FIG. 49 in which A is the aqueous solution A; AT is the tube transporting the aqueous solution A; B is the aqueous solution B; BT is the tube transporting the aqueous solution B; M is the mixing tube; C is the collector; S is the separator; and D is the drier/calcinating oven. The two syringes of the perfusion pumps with cylindrical ends were connected to Masterflex® Tygon Chemical, I/P 26 tubing with an internal diameter of 6.4 mm and Masterflex®Tygon Chemical, I/P 15 tubing with an internal diameter of 4.8 mm for aqueous solutions A and B respectively and were connected by a glass Y-coupling piece under a 120° angle with an internal diameters of 4 mm to aqueous solution A, 3 mm to aqueous solution B and 4 mm to a 180 cm long mixing tube of Masterflex® Tygen Chemical, I/P 26 with an internal diameter of 6.4 mm. The syringe A was filled with aqueous solution 7 and syringe B with aqueous solution 8 and aqueous solution 7 and aqueous solution 8 were jetted into one another at rates of 1130 mL/min (2107.5 m/h) and 420 mL/min (1392.6 m/h) respectively into the 180 cm long mixing tube realizing a pH of 5.0 in the mixture. The linear velocity through the mixing pipe was 2890 m/h and the residence time of the mixture of aqueous solution 1 and aqueous solution 2 in the mixing tube was 2.24 s.

The pumps used were a Cole Palmer Masterflex® “I/P” Precision Brushless Drive, 33 to 650 rpm with I/P “Easy-Load” pump head, PSF housing/SS rotor for solution 7 and a Cole Palmer Masterflex® “L/S” precision variable-speed drive, 6 to 600 rpm; L/S “High-Performance” Pump Head for solution 8 and the pumps were started simultaneously. Upon contact silica condensation was readily visible. The residence time in the 180 cm tube was ca. 3 s. The products produced in the first 10 s were discarded. Particles flowed out through the tubing and were collected in a beaker as a white suspension. Ten minutes after collection the particles recovered were washed 3 times with deionized water the resulting material was first dried at 60° C. overnight and then calcined in an oven by heating up at a rate of PC/min to a temperature of 350° C. and then holding the temperature at 350° C. for 8 h.

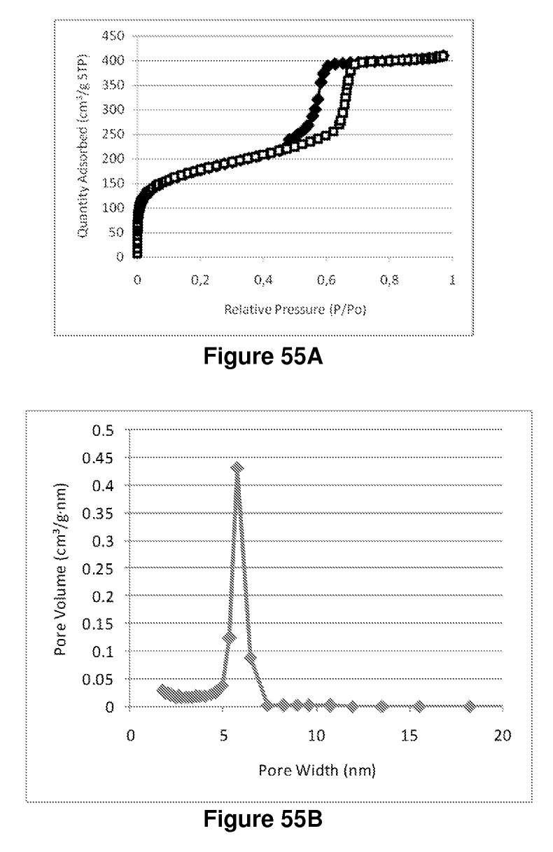

The resulting material was characterized by nitrogen adsorption performed on a Micromeritics Tristar apparatus. Prior to measurement, the sample was pretreated at 200° C. for 10 h (ramp: 5° C./min). The isotherm and pore size distribution are shown in FIGS. 55A and 55B respectively. The pore size was 5.8 nm.

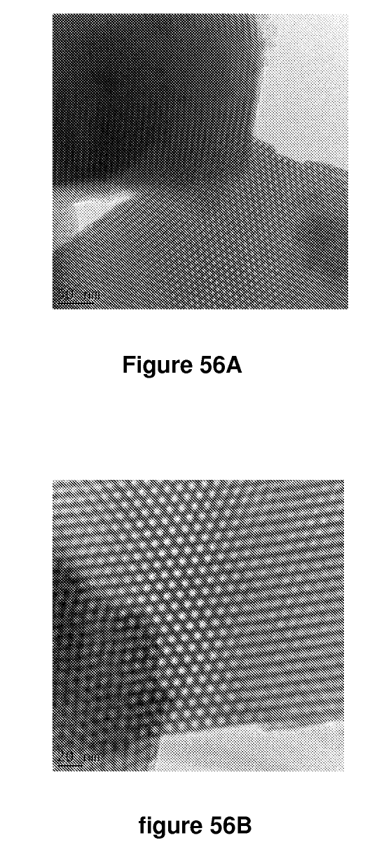

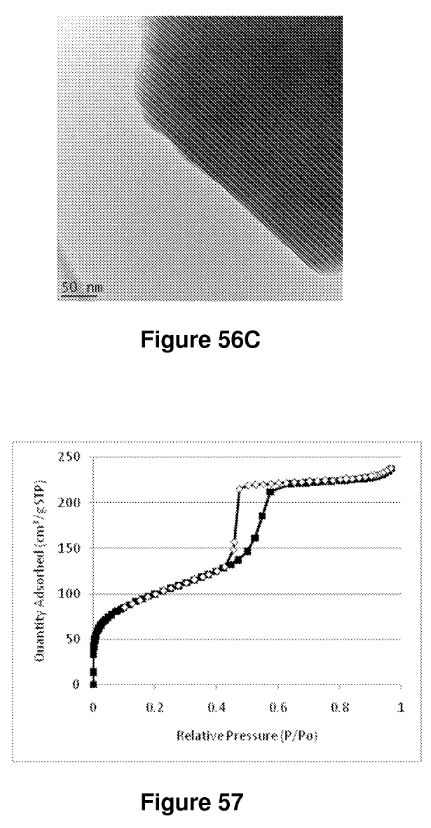

The P6m hexagonal ordering of the mesophase is clearly illustrated in the TEM micrographs of the calcined mesoporous silica shown in FIGS. 56A, 56B and 56C.

### Example 29

Aqueous (silicate precursor) solution 10 was prepared by diluting 10.4 g of sodium silicate solution (10 wt % NaOH, 27 wt % SiO₂) (Merck, Germany) with 30.0 g of deionized water with a pH of 11.0.

Solution 10 was added to solution 9 with stirring at 150 rpm with a mechanical mixer. The stirring was stopped after 1 minute. No further ageing steps were performed. The product was recovered by vacuum filtration, washed with 300 ml of deionised water and then was dried at 60° C. prior to being calcined in an oven by heating up at a rate of 1° C./min to a temperature of 550° C. and holding the temperature at 550° C. for 8 h.

The resulting material was characterized by nitrogen adsorption performed on a Micromeritics Tristar apparatus. Prior to measurement, the sample was pretreated at 200° C. for 10 h (ramp: 5° C./min). The isotherm is shown in FIG. 57.

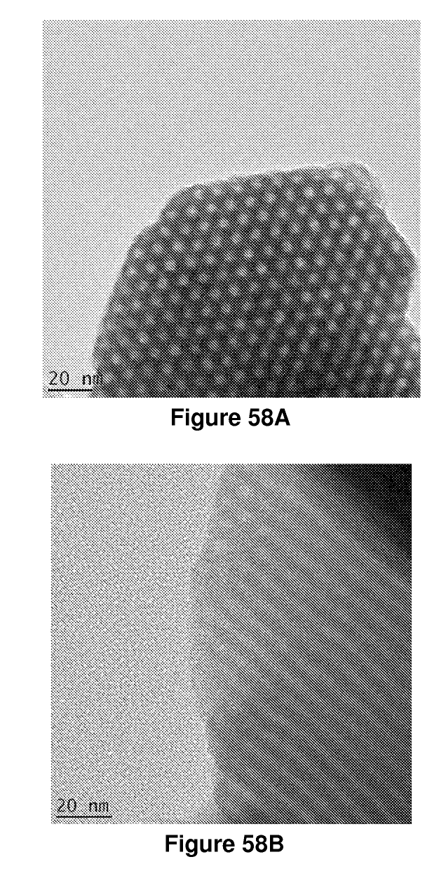

The P6m hexagonal ordering of the mesophase is clearly illustrated in the TEM micrographs of the calcined mesoporous silica shown in FIGS. 58A and 58B.

### Example 30

The materials listed in Table 1 hereinabove were synthesized as follows.

4.0 g of Pluronic™ P123 were dissolved in 107.5 g of water. To this solution, 3.684 g citric acid monohydrate and 2.540 g of trisodium citrate were added. The resulting surfactant solution was stirred for 24 h. 10.4 g sodium silicate solution (10% NaOH, 27% SiO₂) was diluted with 30.0 g of water and added to the surfactant solution. The pH was measured prior to and after sodium silicate addition. The solution was stirred for 5 min at 175 rpm with a mechanical stirrer and kept at room temperature (20° C.) without agitation for 24 h. After this step, an optional heating step at 90° C. for another 24 h was sometimes applied. The as-synthesized material was filtered, washed and dried at 60° C. overnight. Finally the material was calcined in air in two steps, 8 h at 300° C. and 8 h at 500° C. with 1° C. min⁻¹ ramps.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents, patent applications, patent application publications, and other publications cited or referred to in this specification are herein incorporated by reference to the same extent as if each independent patent, patent application, patent application publication or publication was specifically and individually indicated to be incorporated by reference.

What is claimed is:

1. A process for preparing a 2D-hexagonal ordered mesoporous silica material with a substantially uniform pore size in the range of 4 to 30 nm comprising the steps of:
preparing an aqueous solution 1 comprising an alkali silicate solution;
preparing an aqueous solution 3 comprising a poly(alkylene oxide) triblock copolymer and a buffer with a pH greater than 2 and less than 8, said buffer having an acid and a base component;
adding said aqueous alkali silicate solution to said aqueous solution giving a pH greater than 2 and less than 8 and allowing a reaction between the components to take place at a temperature in the range of 10 to 100° C., and
filtering off, drying and calcinating the reaction product to produce said 2D-hexagonal ordered mesoporous silica material with a substantially uniform pore size.

2. The process according to claim 1, wherein said buffer with a pH greater than 2 and less than 8 is a sodium citrate/citric acid buffer, a Na₂HPO₄/citric acid buffer, a HCl/sodium citrate buffer or a Na₂HPO₄/NaH₂PO₄ buffer.

3. The process according to claim 2, wherein said buffer is a sodium citrate/citric acid buffer.

4. The process according to claim 3, wherein the sodium citrate/citric acid buffer has a sodium citrate:citric acid weight ratio in the range of 0.10:1 to 3.3:1.

5. The process according to claim 1, wherein the process is conducted at a pH that is in the range of 5 to 7.

6. The process according to claim 1, wherein said poly(alkylene oxide) triblock copolymer is HO(CH₂CH₂O)₂₀(CH₂CH(CH₃)O)₇₀(CH₂CH₂O)₂₀H (Pluronic P123).

7. The process according to claim 1, wherein the ordered mesoporous silica material has a pore size in the range of 4 to 14 nm.

8. An ordered mesoporous silica material with a substantially uniform pore size in the range of 4 to 30 nm obtainable by a process according to claim 1.

9. A pharmaceutical composition comprising an ordered mesoporous silica material with a substantially uniform pore size in the range of 4 to 30 nm obtainable by a process according to claim 1, and a bioactive species.

10. A process for self-assembling a 2D-hexagonal ordered mesoporous silica material with a substantially uniform pore size in the range of 4 to 30 nm comprising the steps of:
preparing an aqueous solution 1 comprising an alkali silicate solution;
preparing an aqueous solution 4 comprising a poly(alkylene oxide) triblock copolymer and an acid with a pKa in the range 3 to 9;
adding said aqueous solution 1 to said aqueous solution 4 thereby realizing a pH greater than 2 and less than 8 which is within a range of 1.5 pH units above and 1.5 pH units below a pH having the same numerical value as a pKa of said acid with a pKa in the range of 3 to 9 and allowing a reaction between the components to take place at a temperature in the range of 10 to 100° C., and
filtering off, drying and calcinating the reaction product to produce said 2D-hexagonal ordered mesoporous silica material with a substantially uniform pore size.

11. The process according to claim 10, wherein acid with a pKa in the range 3 to 8 is citric acid, acetic acid, succinic acid or phosphoric acid and mixing of aqueous solutions 1 and 4 realizes a citrate/citric acid buffer, an acetate/acetic acid buffer, a succinate/succinic acid buffer or an H₂PO₄/HPO₄⁻ buffer respectively.

12. The process according to claim 10, wherein said aqueous alkali silicate solution is an aqueous sodium silicate solution with at least 10% by weight of sodium hydroxide and at least 27% by weight of silica.

13. A process for producing an ordered mesoporous silica material with a substantially uniform pore size in the range of 4 to 30 nm using a self-assembling method, said process comprising the steps of
preparing an aqueous solution A comprising a silica precursor;
preparing an aqueous solution B, exclusive of an alkali or alkaline earth hydroxide, said aqueous solution B comprising a poly(alkylene oxide) triblock copolymer and an acid with a pKa in the range 3 to 9;
feeding said solution A and said solution B as liquid streams into an elongated mixing receptacle having a first and a second opening such that the liquid streams of solution A and solution B are each discharged independently into said first opening of said elongated mixing receptacle such that they directly impinge thereby giving a pH in the resulting mixture of greater than 2 and less than 8 and producing a reaction product at a temperature in the range of 10 to 100° C. in said elongated mixing receptacle
which upon emergence from said second opening of said elongated mixing receptacle is filtered off, dried and surfactant removal to produce said ordered mesoporous silica material with a substantially uniform pore size;
and wherein said process is exclusive of an ageing step.

14. The process according to claim 13, wherein said silica precursor is an alkali silicate, and said aqueous solution B said aqueous solution B has a pH in the range of 2 to 8.

15. The process according to claim 13, wherein said aqueous solution B further comprises a tetraalkylammonium surfactant that has alkyl groups with 1 to 4 carbon atoms.

16. The process according to claim 13, wherein the residence time in said elongated mixing receptacle is in the range of 1 to 100 s.

17. A 2D-hexagonal ordered mesoporous silica material with a substantially uniform pore size in the range of 4 to 30 nm, with the ratio of Q3 to Q4 silica obtained using ²⁹Si MAS NMR of less than 0.65.

18. The ordered mesoporous silica material according to claim 17, with a substantially uniform pore size in the range of 4 to 14 nm.

19. A 2D-hexagonal ordered mesoporous silica material with a substantially uniform pore size in the range of 4 to 30 nm obtainable according to the process according to claim 1, wherein the ratio of Q3 to Q4 silica obtained using ²⁹Si MAS NMR is less than 0.65.

20. A pharmaceutical composition comprising a 2D-hexagonal ordered mesoporous silica material according to claim 17 and a bioactive species.

21. A process to manufacture a mesoporous material of narrow mesopore size distribution (COK-10) under pH conditions in a self-assembling reaction medium, of which the pH is selected from mildly acidic pH (pH>2) to mildly basic pH (pH<8), as compared to a MCM or SBA framework mesoporous silica material that has been produced under more severe pH conditions in the reaction medium (pH<2 or pH>8).
